(19) United States
(12) Patent Application Publication
(10) Pub. No.: US 2013/0123125 A1
(43) Pub. Date: May 16, 2013

(54) Identification Method of Species of Diatoms in Coast of Korea, and Polynucleotide Probe, DNA Chip and Kit for Identification of Species of Diatoms According to Same

(71) Applicants: Teak-Kyun Lee, Ansung (KR); So-Yun Park, Keoje (KR); Jin-Ik Hwang, Busan (KR); Youn-Ho Lee, Seoul (KR); Man Chang, Seongnam (KR); Gun-Seop Lee, Busan (KR)

(72) Inventors: Teak-Kyun Lee, Ansung (KR); So-Yun Park, Keoje (KR); Jin-Ik Hwang, Busan (KR); Youn-Ho Lee, Seoul (KR); Man Chang, Seongnam (KR); Gun-Seop Lee, Busan (KR)

(73) Assignee: KOREA OCEAN RESEARCH AND DEVELOPMENT INSTITUTE, Anshan, Gyeonggi-do (KR)

(21) Appl. No.: 13/635,887

(22) PCT Filed: Jan. 28, 2011
(86) PCT No.: PCT/KR2011/000592

§ 371 Date: Sep. 18, 2012

(30) Foreign Application Priority Data
Mar. 19, 2010 (KR) 10-2010-0024939

(51) Int. Cl. C12Q 1/68

(52) U.S. Cl. CPC C12Q 1/6895
USPC 506/9; 506/16; 536/24.3

(57) ABSTRACT

Disclosed is a method for identifying the species of diatoms found in the coastal area of Korea, a polynucleotide probe, a DNA chip and a kit used for identifying the diatom species. The method includes obtaining a PCR product by performing a PCR reaction using the DNA extracted from diatoms, binding the PCR product respectively to a probe which is the same as or complementary to one or more of the nucleotide sequences selected from SEQ. ID. NOs: 3˜51, and identifying the species of a given diatom based on the result of the above binding. More importantly, the present invention enables to identify a given diatom species by analyzing the genotypes of various diatom species found in the coastal area of Korea based on their SNPs in a convenient, fast and accurate way.

### TECHNICAL FIELD

The present invention relates to a method of identifying 36 major diatom species having their habitat in the coastal area of Korea. In particular, the present invention relates to a fast and accurate method of identifying diatom species having their habitat in the sea of Korea by the analysis of their genotypes based on single nucleotide polymorphism (SNPs) of various diatoms, a polynucleotide probe, a DNA chip and a kit for identifying the same.

### BACKGROUND ART

Generally, the traditional study on the taxonomy of biological species has been conducted based on metric traits and meristic traits.

However, marine microalgae are known to change their morphological characteristics according to their environmental conditions and thus there have been limitations in their morphological classification. In fact, there is some slight difference among taxonomists with regard to the taxonomy of marine microalgae which causes a problem in monitoring varieties in the ecosystem. Therefore, there has been a need for the development of a technology for analyzing DNA bar code information using a molecular marker and for the accurate identification using a DNA chip. The analysis of DNA bar code information and the variety of marine animals using DNA chips can be used for monitoring future marine environmental conditions and the study on their preservation.

However, regarding the various diatoms present in the coastal area of Korea known as major fishing grounds of Korea, there has been little information or report in the world on the fast and accurate identification of various species of diatoms simultaneously using molecular biological method.

### SUMMARY OF INVENTION

### Technical Problem

The present invention provides a fast and accurate method for identifying the species of major diatoms having their habitat in the coast of Korea based on the analysis of their genotypes.

In classifying a given animal species, where a difference in nucleotide sequence among the species has been known, a standardized fast and accurate method of sequence analysis will enable to reduce the time, labor, and cost involved therein. Therefore, the present invention provides a polynucleotide probe prepared based on the distinct difference in nucleotide sequence among the major 36 marine diatom species found in the coast of Korea. Further, the present invention provides a fast and accurate method for identifying the difference in nucleotide sequence using a DNA chip or a DNA kit including the polynucleotide probe.

According to an exemplary embodiment of the present invention, there is provided a kit comprising probes having 15-30 bp long continuous nucleotide sequences including the domains showing distinct differences in nucleotide sequence among the major 36 marine diatom species, and DNA chips including the probes. The present invention provides a fast and accurate method for identifying the species of major diatoms found in the coast of Korea by the analysis of their genotypes on a slide using the kit.

The inventors of the present invention aims at designing an optimal polynucleotide probe which can species specially bind to each of the above-mentioned diatom species based on the SNPs domain of cytochrome oxidase subunit I (COI) gene among mitochondrial DNAs of various diatom species.

### Technical Solution

In order to achieve the objectives mentioned above, the method for identifying the diatom species according to the present invention includes the steps of obtaining a PCR product by performing a PCR reaction using DNA extracted from diatoms; binding the PCR product respectively to a probe which is the same as or complementary to one or more of the DNA sequences selected from SEQ. ID. NOs: 3˜51; and identifying the species of a given diatom based on the result of the above binding.

In an exemplary embodiment of the present invention, there is provided a probe for identifying diatom species consisting of one or more of polynucleotide which respectively includes a nucleotide sequence being the same as or complementary to one selected from SEQ. ID. NOs: 3˜51. In another exemplary embodiment of the present invention, there is provided a DNA chip for identifying diatom species.

In a further exemplary embodiment of the present invention, there is provided a kit for identifying diatom species including polynucleotide probes having 15-30 bp long continuous nucleotide sequences being the same as or complementary to one or more DNAs selected from SEQ. ID. NOs: 3˜51, which bind to the SNPs domain of COI gene among mitochondrial DNA of diatom species; and a primer for PCR amplification.

Other aspects and preferred embodiments of the invention are discussed infra

### Advantageous Effects of Invention

In the present invention, the genotypes of various diatom species were analyzed. Based on the result, it has become possible to identify the species of major marine diatom species found in the coastal area of Korea in a convenient, fast and accurate way based on the SNPs of the diatoms.

That is, the inventors of the present invention selected COI gene among mitochondrial DNA as a most suitable gene group for the identification of diatom species, discovered the specific SNPs of the diatoms present therein, and arbitrarily prepared DNA sequences based on the same that can help to distinguish various diatom species, thereby enabling a convenient and easy identification of diatom species.

Further, the present invention can be prepared into a probe for the identification of diatom species, a DNA chip or a DNA kit including the same, thereby enabling to identify the diatom species even when they are present in the form of a larva, a seasoned processed product, and powder, which are hardly discernible of its species, simply by placing the specimen on the top of a slide.

In addition, by using the probe prepared according to the present invention, can considerably reduce the time required for the analysis of a given specimen by utilizing microarray method compared with the conventional method, thereby enabling to conduct a large number of specimens in a relatively short time.

### BRIEF DESCRIPTION OF DRAWINGS

FIGS. 1 to 36 respectively show diagrams sequentially illustrating the nucleotide sequences of the SNPs including domains of COI gene in the mitochondrial DNA of the 36 major diatom species according to exemplary embodiments of the present invention.

FIG. 37 is a diagram illustrating a structure of a DNA chip for the identification of diatom species including oligonucleotide probes prepared based on the SNPs shown in FIGS. 1 to 36 according to an exemplary embodiment of the present invention.

FIGS. 38 to 54 are pictures respectively showing the results of binding between the oligonucleotide probes and PCR amplified products of certain diatom species including Achanathes longipes by using the DNA chip shown in FIG. 37 according to exemplary embodiments of the present invention.

### DESCRIPTION OF EMBODIMENTS

Hereinafter, a technical construction of the present invention will be described with reference to the accompanying drawings.

In an embodiment of the present invention, there is provided a method for identifying diatom species including extracting DNA from various marine specimens that belong to diatoms and perform PCR reaction using thus obtained DNA thereby obtaining PCR amplified products.

In extracting DNA from the above-mentioned specimens, the specimens may include any tissues of diatoms or their processed products from which a given species can be identified based on its DNA analysis. Therefore, there is no limitation on from which of the specimen the tissue is collected for DNA extraction.

Further, PCR amplification of thus obtained DNA extract is to increase specimens for examination and thus there is no limitation regarding the method of PCR amplification. That is, any method of DNA extraction commonly used by a person in the art or PCR amplification may be used as necessary.

The inventors of the present invention selected COI gene among mitochondrial DNA as a most suitable gene group for the identification of diatom species, discovered the specific SNPs domains of the diatoms present therein, and based on the same synthesized polynucleotide probes that can species-specifically bind to each diatom species. Because these polynucleotide probes were synthesized based on the SNPs domains of the relevant diatom species they can only bind to the specific DNA of the very species but not to other unrelated DNAs.

In particular, the inventors of the present invention, after extensive study and research efforts, confirmed that those DNA sequences which correspond to SEQ. ID. NOs.: 3˜51 of COI gene among mitochondrial DNA of the 36 diatom species having their habitat in the coastal area of Korea are the most suitable gene group for the identification of diatom species. In addition, they also confirmed that by using the polynucleotide probes which are either the same as or complementary to the DNA sequences of the above-mentioned SEQ. ID. NOs.: 3˜51 are able to identify with excellence a given diatom species.

Although the subject matter of the present invention is not limited to any specific animal species, it is preferred that the species be a diatom species found in the coastal area of Korea, and more preferably a diatom species in the southern coastal area of Korea. As referred to herein, “Korea” or “the coastal area of Korea” are the sea adjacent to the territory of Republic of Korea which forms the borderline (coast guard or shore line) between the sea and the land. In addition, “southern sea” or “southern coastal area” refer to the sea located in the southern part of Republic of Korea, in general, to Tsushima island in East, Heuksando in West, and Jeju island in South.

The present invention particularly uses SNPs domain present in the COI gene of mitochondrial DNA. Accordingly, the DNA extracted from the above diatom species are preferred to include the SNPs in the COI gene of mitochondrial DNA of the diatom species.

As referred to herein, “polymorphism” is a two or more of general sequences or allelomorphic characters present within a genetically determined group. A polymorphic marker or position is the locus where divergence occurs. A desirable marker must have at least two allelomorphic characters with 1% or greater of a frequency of occurrence in a selected group, more preferably 10% or 20% or greater. A polymorphic domain can be a single base pair.

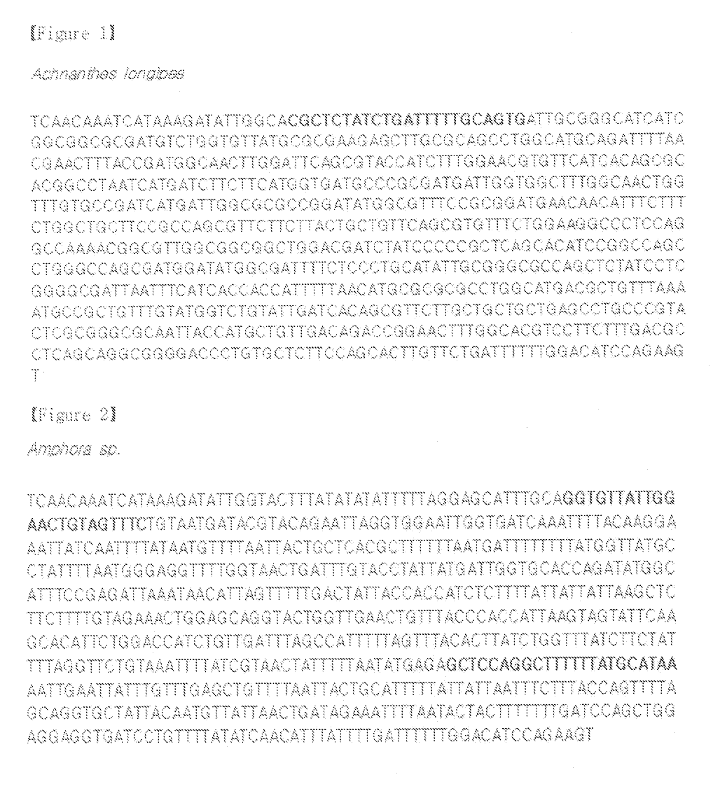

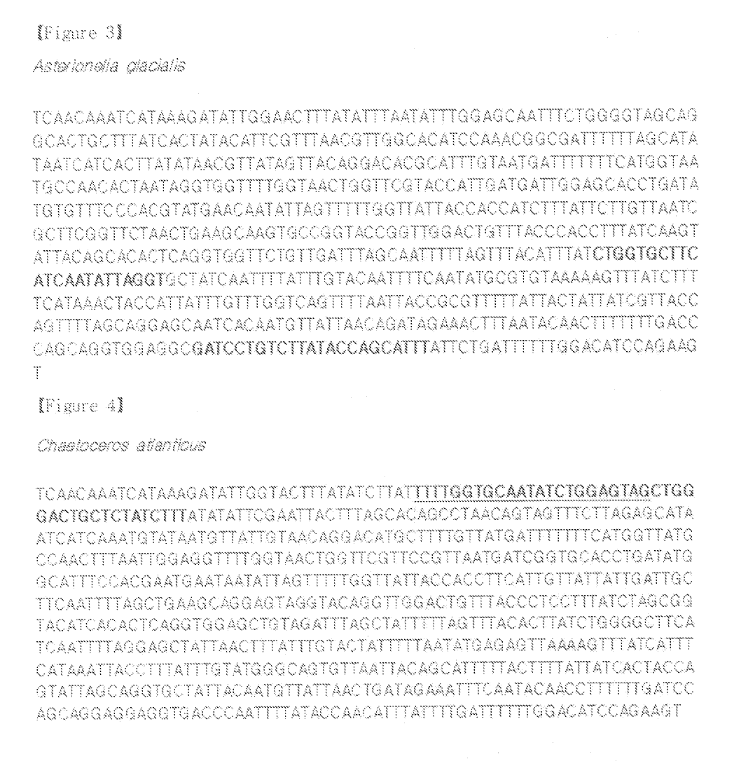

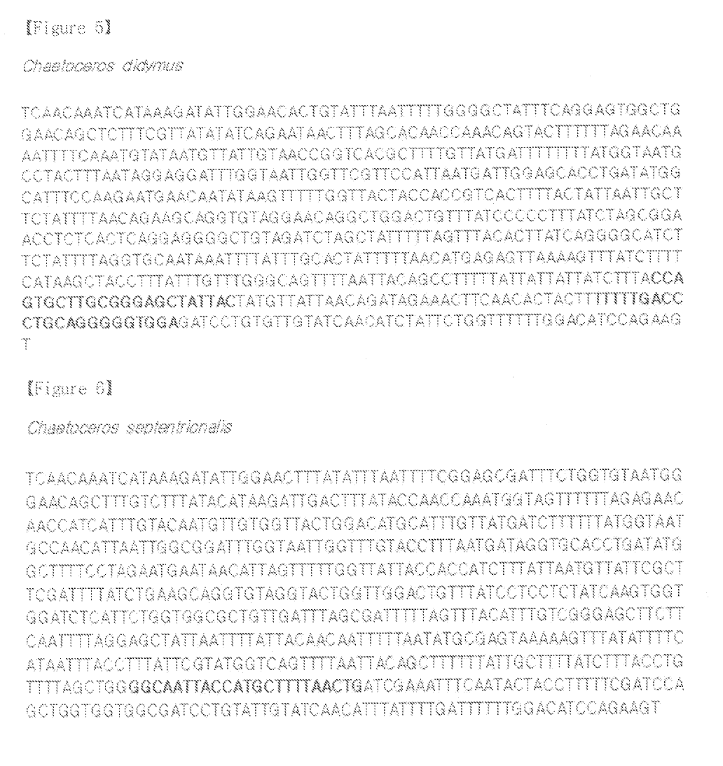

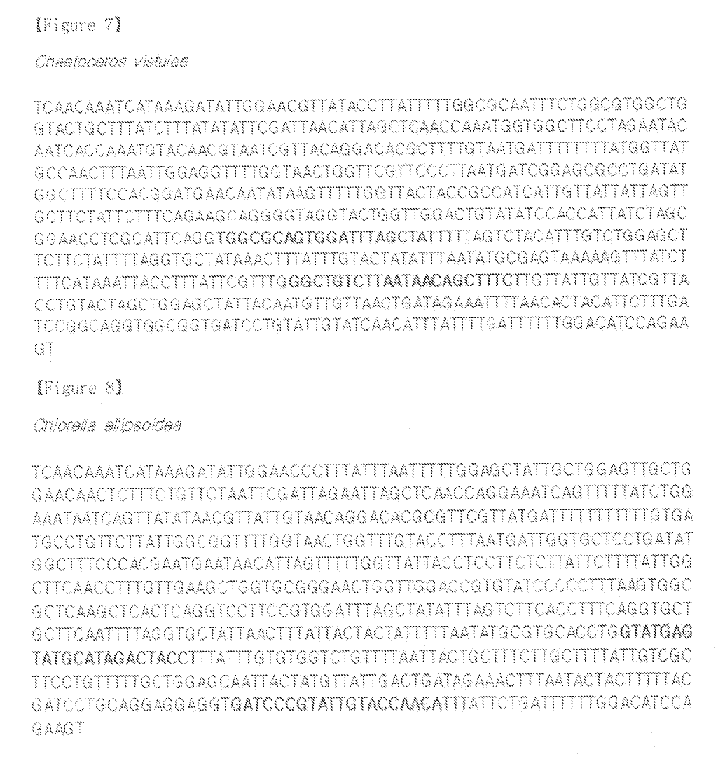

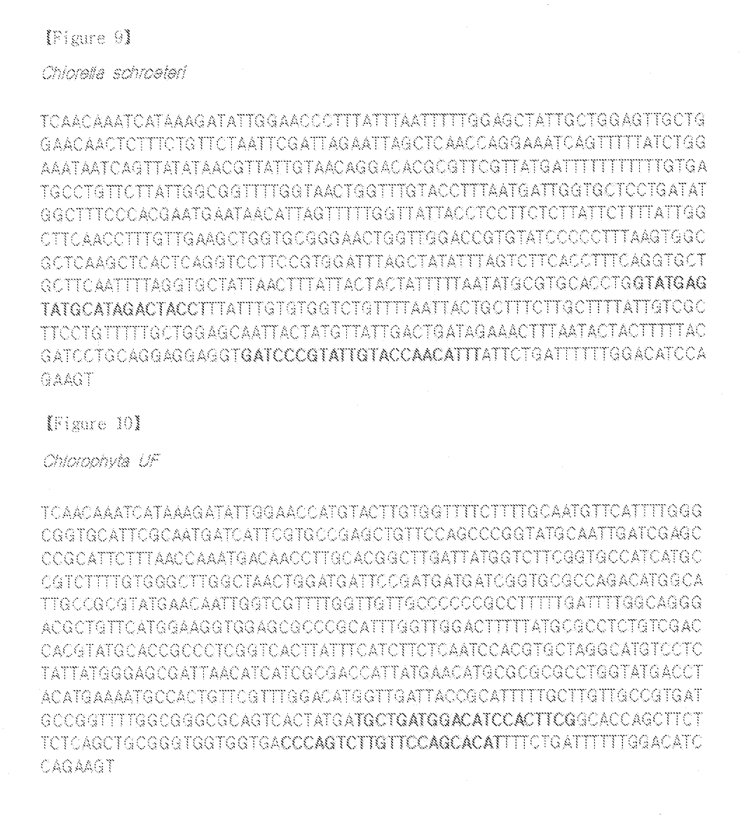

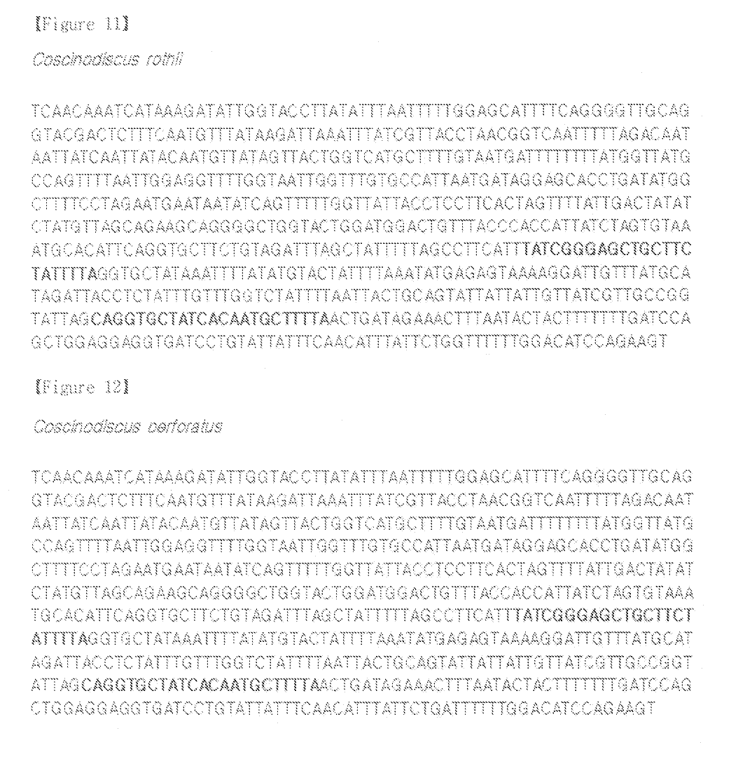

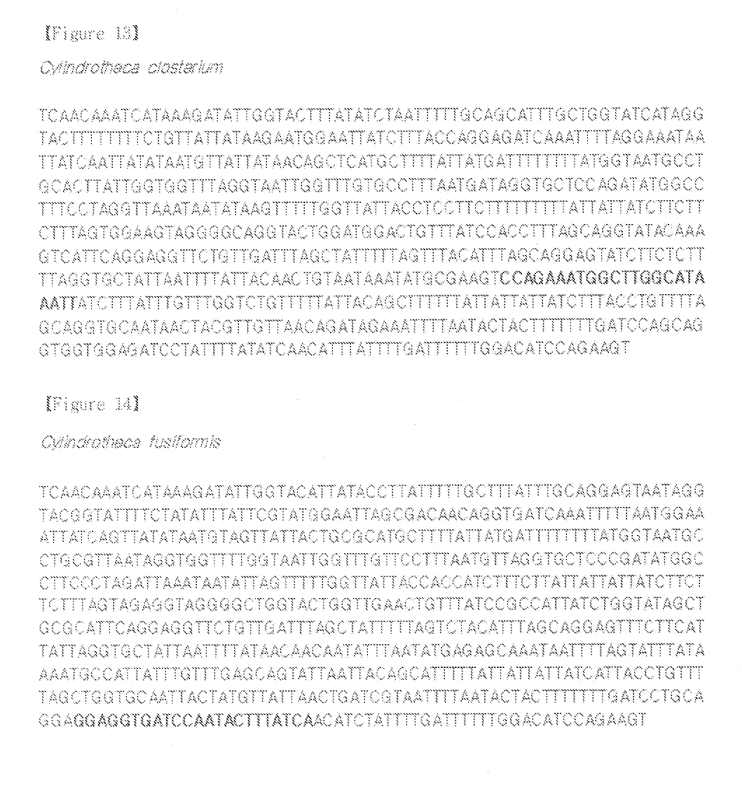

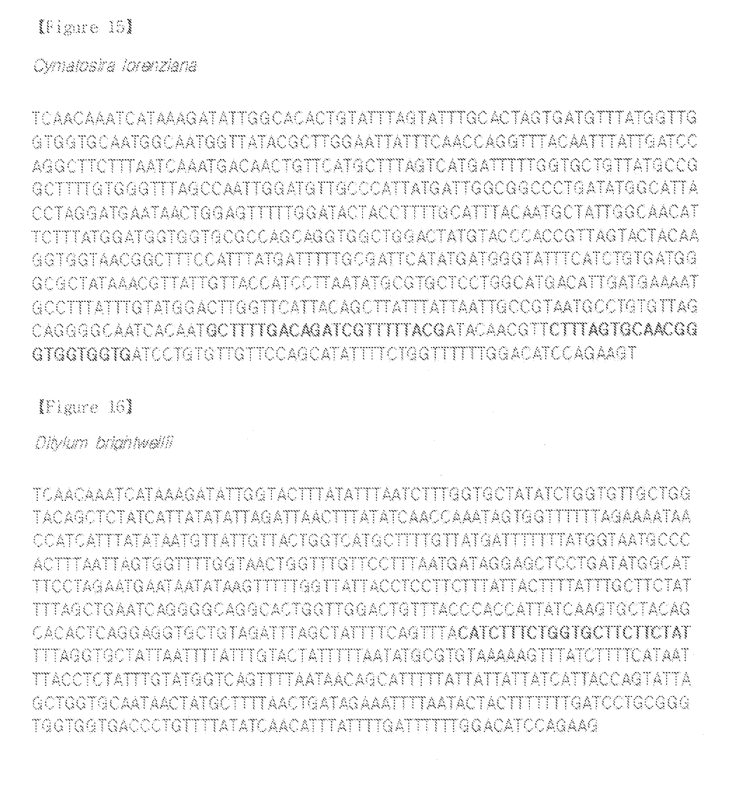

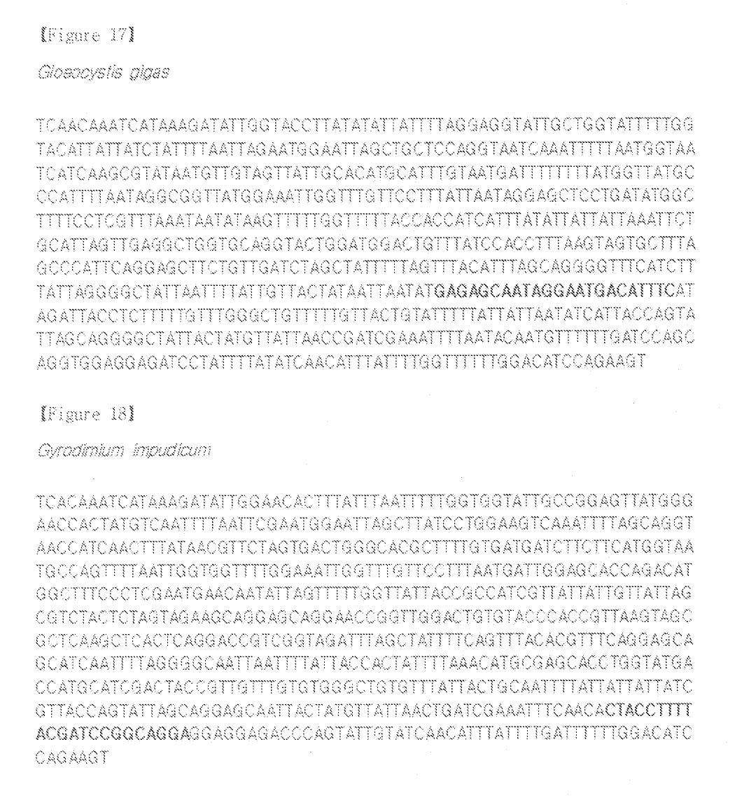

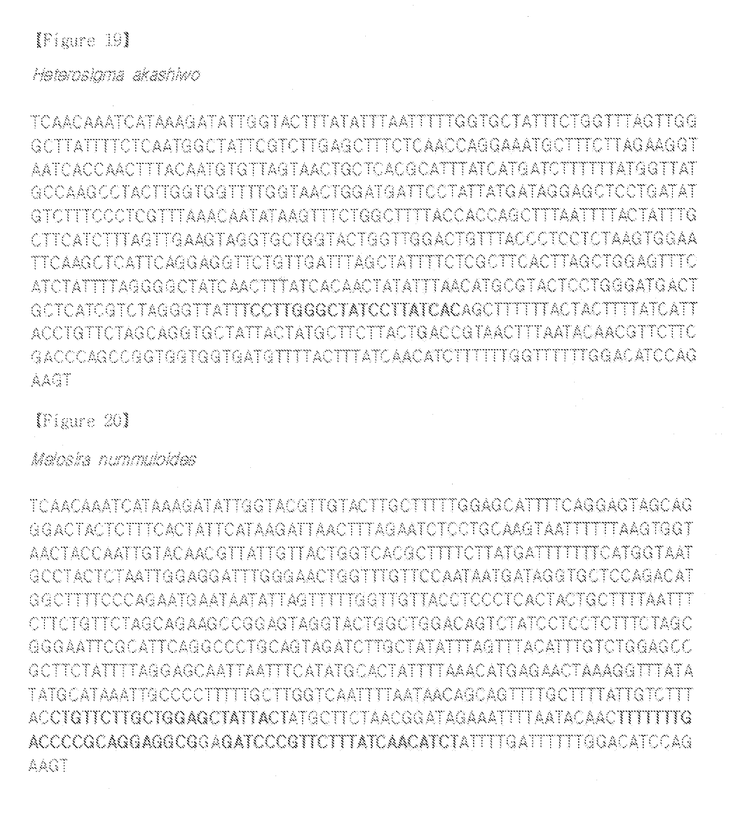

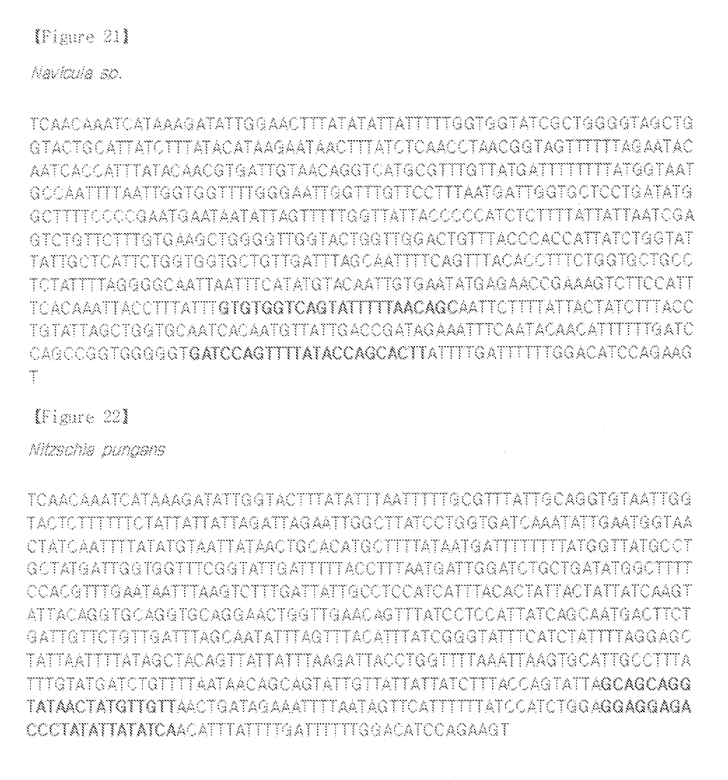

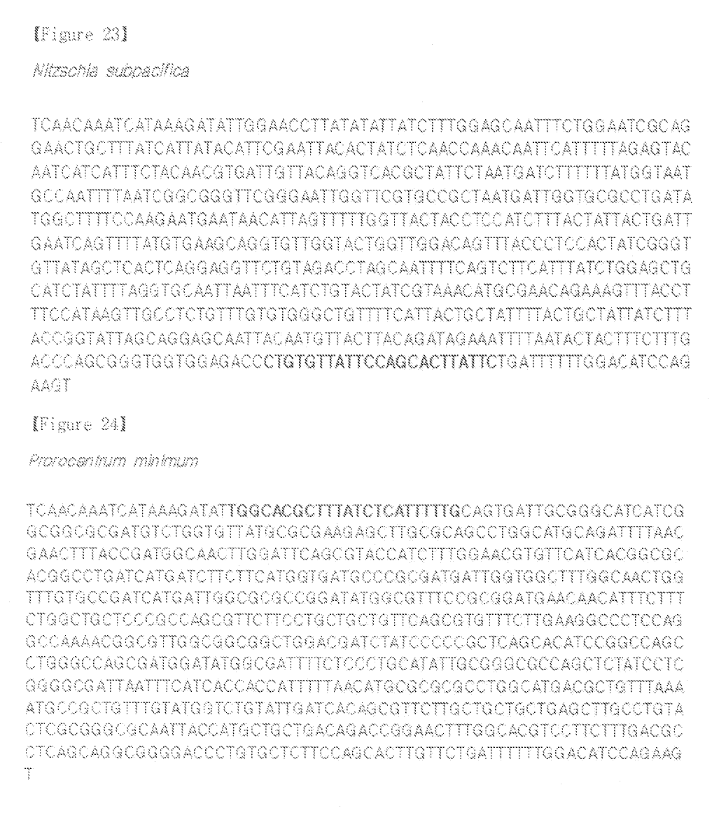

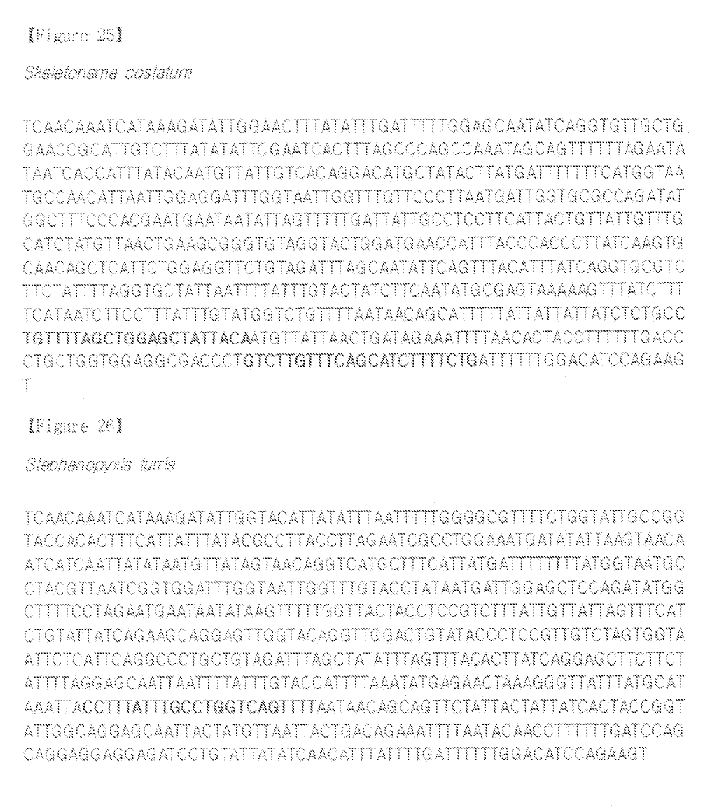

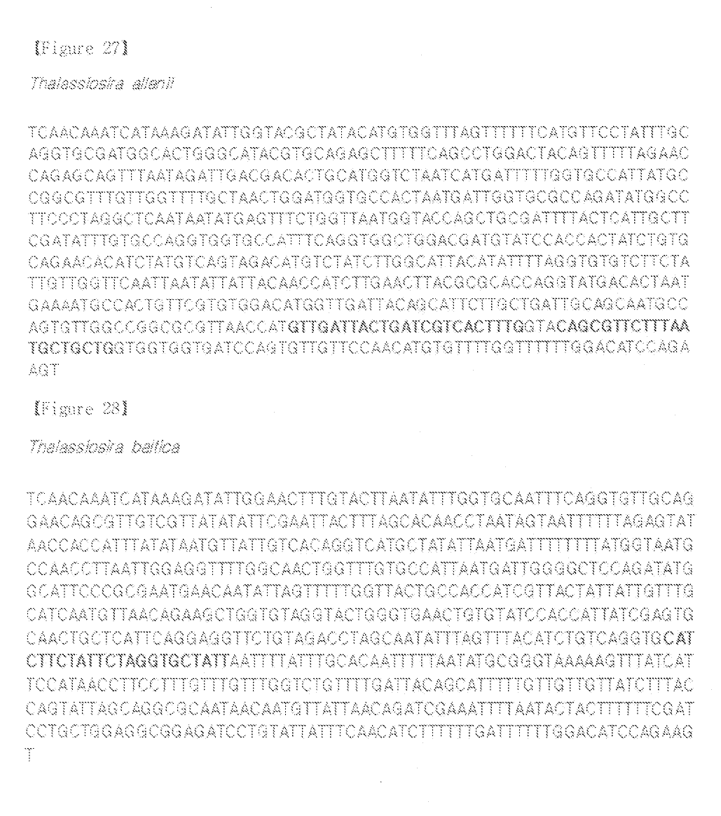

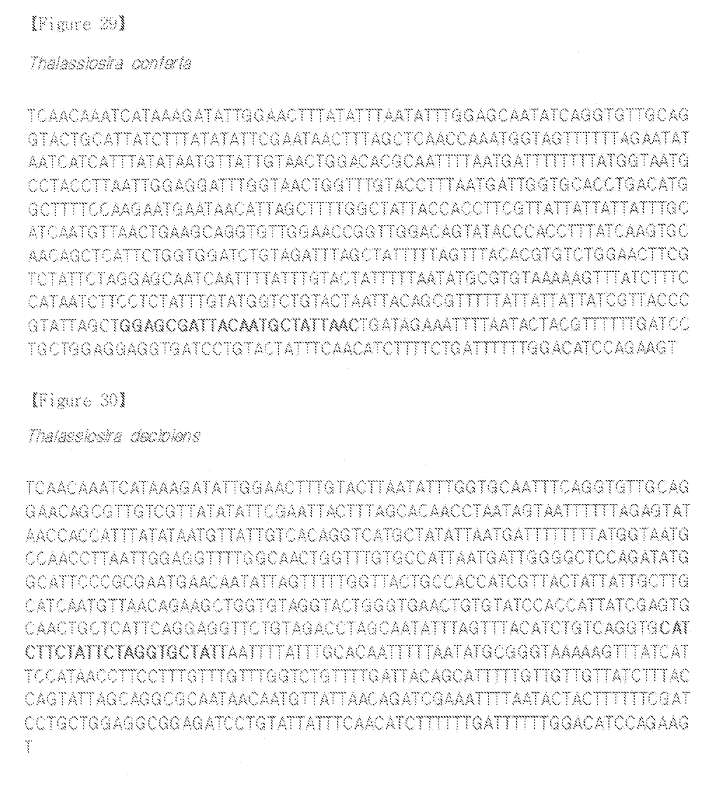

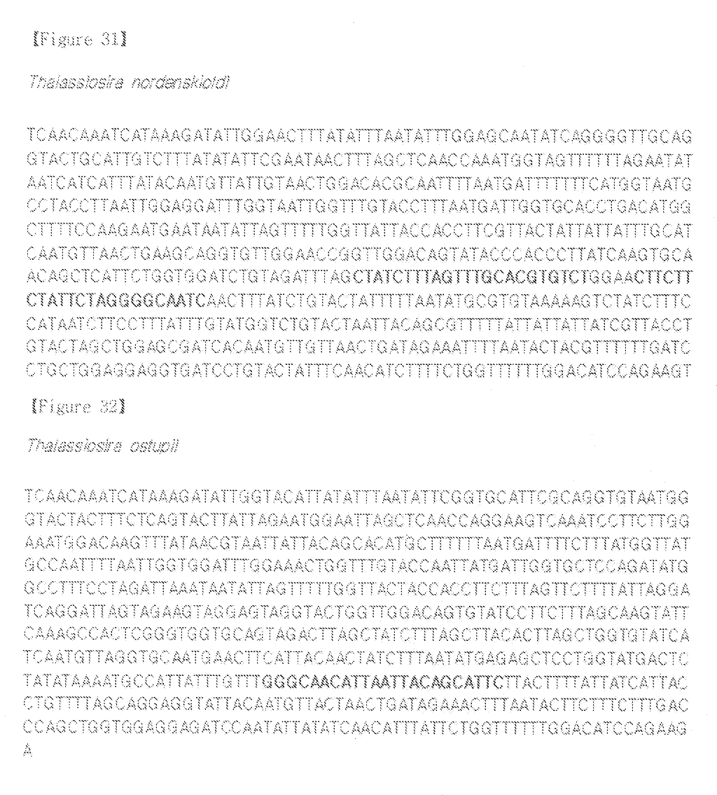

FIGS. 1 to 36 respectively show diagrams sequentially illustrating the nucleotide sequences of the SNPs including domains of COI gene in the mitochondrial DNA of the 36 major diatom species.

The inventors of the present invention confirmed that, after extensive study and research efforts, confirmed that it is desirable to use the nucleotide sequence that corresponds to the SNPs in the. COI gene of hairtail DNA as a probe to identify a given species of diatoms.

Accordingly, the present invention employs as a probe a nucleotide sequence which is the same as or complementary to a nucleotide sequence that belongs to SNPs domain, i.e., at least one DNA sequence selected from SEQ. ID. NOs.: 3˜51. These probes were synthesized based on the SNPs domain which differs from diatom to diatom. Therefore, these probes should only bind to the PCR product amplified using an intended diatom specimen but not to the one amplified using a different diatom species.

The DNA sequences of SEQ. ID. NOs.: 3˜51 according to the present invention are shown in Table 1 below.

TABLE 1
DNA sequences for the identification of major diatom species found
in the coastal area of Korea

| | | | Position of |
|---|---|---|---|
| Name of Probe | DNA sequence | Diatom Species | DNA sequence |
| B1(SEQ. ID. NO. 3) | CACTGCAAAAATCA | Achnanthes longipes | 26-49 |
| | GATAGAGCG | | |
| B2(SEQ. ID. NO. 4) | GCTCCAGGCTTTTT | Amphora sp. | 544-567 |
| | TATGCATAA | | |
| B3(SEQ. ID. NO. 5) | GAAACTACAGTTCC | Amphora sp. | 55-78 |
| | AATAACACC | | |
| B4(SEQ. ID. NO. 6) | GATCCTGTCTTATA | Asterionella glacialis | 706-729 |
| | CCAGCATTT | | |
| B5(SEQ. ID. NO. 7) | CTGGTGCTTCATCA | Asterionella glacialis | 485-508 |
| | ATATTAGGT | | |
| B6(SEQ. ID. NO. 8) | AAAGATAGAGCAGT | Chaetoceros atlanticus | 58-81 |
| | CCCAGCTAC | | |
| B7(SEQ. ID. NO. 9) | CTACTCCAGATATT | Chaetoceros atlanticus | 39-62 |
| | GCACCAAAA | | |
| B8(SEQ. ID. NO. 10) | TTTTTGACCCTGCA | Chaetoceros didymus | 638-706 |
| | GGGGGTGGA | | |
| B9(SEQ. ID. NO. 11) | CCAGTGCTTGCGG | Chaetoceros didymus | 625-648 |
| | GAGCTATTAC | | |
| B10(SEQ. ID. NO. 12) | GGCAATTACCATG | Chaetoceros septentrionalis | 639-662 |
| | CTTTTAACTG | | |
| B11(SEQ. ID. NO. 13) | GGCTGTCTTAATA | Chaetoceros vistulae | 585-608 |
| | ACAGCTTTCT | | |
| B12(SEQ. ID. NO. 14) | TGGCGCAGTGGAT | Chaetoceros vistulae | 447-470 |
| | TTAGCTATTT | | |
| B13(SEQ. ID. NO. 15) | GATCCCGTATTGT | Chlorella ellipsoidea, C. | 706-729 |
| | ACCAACATTT | schroeteri | |
| B14(SEQ. ID. NO. 16) | GTATGAGTATGCA | Chlorella ellipsoidea, C. | 551-574 |
| | TAGACTACCT | schroeteri | |
| B15(SEQ. ID. NO. 17) | CCCAGTCTTGTTC | Chlorophyta UF | 709-730 |
| | CAGCACAT | | |
| B16(SEQ. ID. NO. 18) | TGCTGATGGACAT | Chlorophyta UF | 654-675 |
| | CCACTTCG | | |
| B17(SEQ. ID. NO. 19) | CAGGTGCTATCAC | Coscinodiscus perforatus, C. | 635-658 |
| | AATGCTTTTA | rothii | |
| B18(SEQ. ID. NO. 20) | TATCGGGAGCTGC | Coscinodiscus perforatus, C. | 482-505 |
| | TTCTATTTTA | rothii | |
| B19(SEQ. ID. NO. 21) | CCAGAAATGGCTT | Cylindrotheca closterium | 547-570 |
| | GGCATAAATT | | |
| B20(SEQ. ID. NO. 22) | AAATATGCGAAGT | Cylindrotheca closterium | 534-557 |
| | CCAGAAATGG | | |
| B21(SEQ. ID. NO. 23) | GGAGGTGATCCAA | Cylindrotheca fusiformi | 700-723 |
| | TACTTTATCA | | |
| B22(SEQ. ID. NO. 24) | CTTTAGTGCAACG | Cymatosira lorenziana | 684-704 |
| | GGTGGTGGTG | | |
| B23(SEQ. ID. NO. 25) | GCTTTTGACAGAT | Cymatosira lorenziana | 651-674 |
| | CGTTTTTACG | | |
| B24(SEQ. ID. NO. 26) | CATCTTTCTGGTG | Ditylum brightwellii | 478-501 |
| | CTTCTTCTAT | | |
| B25(SEQ. ID. NO. 27) | GAGAGCAATAGGA | Gloeocystis gigas | 540-563 |
| | ATGACATTTC | | |
| B26(SEQ. ID. NO. 28) | CTACCTTTTACGA | Gyrodimium impudicum | 677-700 |
| | TCCGGCAGGA | | |
| B27(SEQ. ID. NO. 29) | TCCTTGGGCTATC | Heterosigma akashiwo | 580-601 |
| | CTTATCAC | | |
| B28(SEQ. ID. NO. 30) | GATCCCGTTCTTT | Melosira nummuloides | 707-729 |
| | ATCAACATCT | | |
| B29(SEQ. ID. NO. 31) | TTTTTTTGACCCC | Melosira nummuloides | 681-704 |
| | GCAGGAGGCG | | |
| B30(SEQ. ID. NO. 32) | CTGTTCTTGCTGG | Melosira nummuloides | 626-649 |
| | AGCTATTACT | | |
| B31(SEQ. ID. NO. 33) | GATCCAGTTTTAT | Navicula sp. | 706-729 |
| | ACCAGCACTT | | |
| B32(SEQ. ID. NO. 34) | GTGTGGTCAGTAT | Navicula sp. | 580-603 |
| | TTTTAACAGC | | |
| B33(SEQ. ID. NO. 35) | GGAGGAGACCCTA | Nitzschia pungens | 100-723 |
| | TATTATATCA | | |
| B34(SEQ. ID. NO. 36) | GCAGCAGGTATAA | Nitzschia pungens | 634-657 |
| | CTATGTTGTT | | |
| B35(SEQ. ID. NO. 37) | CTGTGTTATTCCA | Nitzschia subpacifica | 710-733 |
| | GCACTTATTC | | |
| B36(SEQ. ID. NO. 38) | CAAAAATGAGATA | Prorocentrum minimum | 21-44 |
| | AAGCGTGCCA | | |
| B37(SEQ. ID. NO. 39) | GTCTTGTTTCAGC | Skeletonema costatum | 712-734 |
| | ATCTTTTCTG | | |
| B38(SEQ. ID. NO. 39) | CTGTTTTAGCTGG | Skeletonema costatum | 626-649 |
| | AGCTATTACA | | |
| B39(SEQ. ID. NO. 41) | CCTTTATTTGCCT | Stephanopyxis turris | 571-594 |
| | GGTCAGTTTT | | |
| B40(SEQ. ID. NO. 41) | CAGCGTTCTTTAA | Thalassiosira allenii | 678-699 |
| | TGCTGCTG | | |
| B41(SEQ. ID. NO. 43) | GTTGATTACTGAT | Thalassiosira allenii | 651-674 |
| | CGTCACTTTG | | |
| B42(SEQ. ID. NO. 44) | CATCTTCTRTTCT | Thalassiosira baltica. | 491-514 |
| | AGGTGCTATT | T. decepiens, T. puntigera | |
| B43(SEQ. ID. NO. 45) | GGAGCGATTACAA | Thalassiosira conferta | 637-660 |
| | TGCTATTAAC | | |
| B44(SEQ. ID. NO. 46) | CTTCTTCTATTCT | Thalassiosira nordenskioldi, | 491-514 |
| | AGGGGCAATC | T. weissflogii | |
| B45(SEQ. ID. NO. 47) | CTATCTTTAGTTT | Thalassiosira nordenskioldi, | 464-487 |
| | GCACGTGTCT | T. weissflogii | |
| B46(SEQ. ID. NO. 48) | CTTCTTCTATTCT | Thalassiosira nordenskioldi, | 491-514 |
| | AGGGGCAATC | T. weissflogii | |
| B47(SEQ. ID. NO. 49) | GGGCAACATTAAT | Thalassiosira ostupii | 584-607 |
| | TACAGCATTC | | |
| B48(SEQ. ID. NO. 50) | TTAACGGGACAAA | Thalassiosira rotula | 242-265 |
| | TCAGTTACCA | | |
| B49(SEQ. ID. NO. 51) | GGTTCTGTAGACT | Thalassiosira rotula | 448-471 |
| | TAGCGATATT | | |

The present invention provides a large number of probes so that they can respectively include those nucleotide sequences which are either the same as or complementary to at least more than one selected from the DNA sequences of SEQ. ID. NOs.: 3˜51 shown above. By allowing at least one of these probes to the PCR products obtained above, and analyzing the result of the binding the species of a given diatom can be identified.

In the present invention, the above diatom species can be one or more selected from the group consisting of Achnanthes longipes, Amphora sp., Asterionella glacialis, Chaetoceros atlanticus, Chaetoceros didymus, Chaetoceros septentrionalis, Chaetoceros vistulae, Chlorella ellipsoidea, C. schroeteri, Chlorophyta UF, Coscinodiscus perforatus, C. rothii. Cylindrotheca closterium, Cylindrotheca fusiformis, Cymstosira lorenziana, Ditylum brightwellii, Gloeocytis gigas, Gyrodimium impudicum, Heterosigma akashiwo, Melosira numnuloides, Navicula sp., Nitzschia pungens, Nitzschia subpacifica, Prorocentrum minimum, Skeletonema costatum, Stephanopyxis turris, Thalassiosira allenii, Thalassiosira baltica. T. decepiens, T. puntigera, Thalassiosira conferta, Thalassiosira nordenskioldi, T. weissflogii, Thalassiosira ostupii, and Thalassiosira rotula

The method of identifying the diatom species according to the binding result is as follows; for example, if a PCR product of a given diatom species binds to the probe of SEQ. ID. NO. 3 the diatom is Achnanthes longipes, if it binds to the probe of SEQ. ID. NO. 4 and/or SEQ. ID. NO. 5, it is Amphora sp. Likewise, other diatom species can be also identified according to the result of the binding.

Further, the probes constructed according to the present invention are preferred that they bind to SNPs domain of COI gene of the mitochondrial DNA of diatom species, and the above binding distinctively differ among on the species.

The above difference in binding specificity is, for example, that the probe including SEQ. ID. NO.: 3 binds specifically to 26^{th}-49^{th} DNA sequence of COI gene of mitochondria DNA of Achnanthes longipes, the probe including SEQ. ID. NO.: 4 to 15544^{th}-577^{th} DNA sequence of Amphora sp., the probe including SEQ. ID. NO.: 5 to 55^{th}-78^{th} DNA sequence of Amphora sp., the probe including SEQ. ID. NO.: 6 to 706^{th}-729^{th} DNA sequence of Asterionella glacialis, the probe including SEQ. ID. NO.: 7 to 485^{th}-508^{th} DNA sequence of Asterionella glacialis, the probe including SEQ. ID. NO.: 8 to 58^{th}-81^{st} DNA sequence of Chaetoceros atlanticus, the probe including SEQ. ID. NO.: 9 to 39^{th}-62^{nd} DNA sequence of Chaetoceros atlanticus, the probe including SEQ. ID. NO.: 10 to 638^{th}-706^{th} DNA sequence of Chaetoceros didymus, the probe including SEQ. ID. NO.: 11 to 625^{th}-648^{th} DNA sequence of Chaetoceros didymus, the probe including SEQ. ID. NO.: 12 to 639^{th}-662^{nd} DNA sequence of Chaetoceros septentrionalis, the probe including SEQ. ID. NO.: 13 to 585^{th}-608^{th} DNA sequence of Chaetoceros vistulae, the probe including SEQ. ID. NO.: 14 to 447^{th}-470^{th} DNA sequence of Chaetoceros vistulae, the probe including SEQ. ID. NO.: 15 to 706^{th}-729^{th} DNA sequence of Chlorella ellipsoidea and C. schroeteri, the probe including SEQ. ID. NO.: 16 to 551^{st}-574^{th} DNA sequence of Chlorella ellipsoidea and C. schroeteri, the probe including SEQ. ID. NO.: 17 to 709^{th}-730^{th} DNA sequence of Chlorophyta UF, the probe including SEQ. ID. NO.: 18 to 654^{th}-675^{th} DNA sequence of Chlorophyta UF, the probe including SEQ. ID. NO.: 19 to 635^{th}-658^{th} DNA sequence of Coscinodiscus perforatus and C. rothii., the probe including SEQ. ID. NO.: 20 to 482^{nd}-505^{th} DNA sequence of Coscinodiscus perforatus and C. rothii., the probe including SEQ. ID. NO.: 21 to 547^{th}-570^{th} DNA sequence of Cylindrotheca closterium, the probe including SEQ. ID. NO.: 22 to 534^{th}-557^{th} DNA sequence of Cylindrotheca closterium, the probe including SEQ. ID. NO.: 23 to 700^{th}-723^{th} DNA sequence of Cylindrotheca fusiformi, the probe including SEQ. ID. NO.: 24 to 684^{th}-704^{th} DNA sequence of Cymstosira lorenziana, the probe including SEQ. ID. NO.: 25 to 651^{st}-674^{th} DNA sequence of Cymstosira lorenziana, the probe including SEQ. ID. NO.: 26 to 478^{th}-501^{st} DNA sequence of Ditylum brightwellii, the probe including SEQ. ID. NO.: 27 to 540^{th}-563^{th} DNA sequence of Gloeocytis gigas, the probe including SEQ. ID. NO.: 28 to 677^{th}-700^{th} DNA sequence of Gyrodimium impudicum, the probe including SEQ. ID. NO.: 29 to 580^{th}-601^{st} DNA sequence of Heterosigma akashiwo, the probe including SEQ. ID. NO.: 30 to 707^{th}-729^{th} DNA sequence of Melosira numnuloides, the probe including SEQ. ID. NO.: 31 to 681^{st}-704^{th} DNA sequence of Melosira numnuloides, the probe including SEQ. ID. NO.: 32 to 626^{th}-649^{th} DNA sequence of Melosira numnuloides, the probe including SEQ. ID. NO.: 33 to 706^{th}-729^{th} DNA sequence of Navicula sp., the probe including SEQ. ID. NO.: 34 to 580^{th}-603^{th} DNA sequence of Navicula sp., the probe including SEQ. ID. NO.: 35 to 100^{th}-723^{rd} DNA sequence of Nitzschia pungens, the probe including SEQ. ID. NO.: 36 to 634^{th}-657^{th} DNA sequence of Nitzschia pungens, the probe including SEQ. ID. NO.: 37 to 710^{th}-733^{rd} DNA sequence of Nitzschia subpacifia, the probe including SEQ. ID. NO.: 38 to 21^{st}-44^{th} DNA sequence of Prorocentrum minimum, the probe including SEQ. ID. NO.: 39 to 712^{th}-734^{th} DNA sequence of Skeletonema costatum, the probe including SEQ. ID. NO.: 40 to 626^{th}-649^{th} DNA sequence of Skeletonema costatum, the probe including SEQ. ID. NO.: 41 to 571^{st}-594^{th} DNA sequence of Stephanopyxis turris, the probe including SEQ. ID. NO.: 42 to 678^{th}-699^{th} DNA sequence of Thalassiosira allenii, the probe including SEQ. ID. NO.: 43 to 651^{st}-674^{th} DNA sequence of Thalassiosira allenii, the probe including SEQ. ID. NO.: 44 to 491^{st}-514^{th} DNA sequence of Thalassiosira baltica. T. decepiens and T. puntigera, the probe including SEQ. ID. NO.: 45 to 637^{st}-660^{th} DNA sequence of Thalassiosira conferta, the probe including SEQ. ID. NO.: 46 to 491^{st}-514^{th} DNA sequence of Thalassiosira nordenskioldi and T. weissflogii, the probe including SEQ. ID. NO.: 47 to 464^{th}-487^{th} DNA sequence of Thalassiosira nordenskioldi and T. weissflogii, the probe including SEQ. ID. NO.: 48 to 491^{st}-514^{th} DNA sequence of Thalassiosira nordenskioldi and T. weissflogii, the probe including SEQ. ID. NO.: 49 to 584^{st}-607^{th} DNA sequence of Thalassiosira ostupii, the probe including SEQ. ID. NO.: 50 to 242^{nd}-265^{th} DNA sequence of Thalassiosira rotula, and the probe including SEQ. ID. NO.: 51 to 448^{th}-471^{th} DNA sequence of Thalassiosira rotula.

Further, the step of binding between a given PCR product above and a probe is preferably performed more than two times. By repeating the binding step before identifying a given diatom species, the above intended binding between the PCR product and the probe can be more solidified.

In another embodiment of the present invention, in identifying a given diatom species, the above PCR reaction is performed so that a polynucleotide being either the same as or complementary to the respective DNA sequences of SEQ. ID. NO.1 or SEQ. ID. NO.2 can be used as a forward direction primer or a reverse direction primer.

That is, in collecting DNA specimens from the 36 major diatom species and amplifying the DNAs via PCR amplification according to the present invention, specific nucleotide sequences suitable for the above diatom species are used as primers. These specific primers correspond to the DNA sequence of the SNP domain of COI gene of the mitochondrial DNA of the diatom species, and the above collected DNAs can be effectively amplified. To this end, the DNAs extracted from blood, cells or tissues as stated above are preferred to include the SNP domain of the COI gene.

For example, as for the species that belong to the above diatoms, it is desirable that the primer including the nucleotide sequences of SEQ. ID. NO.1 and SEQ. ID. NO.2 be used as a forward direction primer and a reverse direction primer.

TABLE 2
Primer sequences for the identification of
diatom species

| Primer | Nucleotide Sequence |
|---|---|
| Forward Primer | TCAACAAATCATAAAGATATTGG |
| (SEQ. ID. NO. 1) | |
| Reverse Primer | ACTTCTGGATGTCCAAAAAAYCA |
| (SEQ. ID. NO. 2) | |

As shown above, the present invention is characterized in that it uses probes which are respectively the same as or complementary to at least one DNA sequences selected from SEQ. ID. NOs.: 3˜51. Accordingly, in an exemplary embodiment of the present invention, there is provided a DNA chip or kit including the probes. In another exemplary embodiment of the present invention, there is further provided a fixed position marker in the above DNA chip and kit, indicated by a specific nucleotide sequence so as to increase the binding to a probe and the accuracy of identification of a diatom species.

In a further exemplary embodiment of the present invention, a large number of diatom species can be identified simultaneously on a single slide according to the DNA microarray technology. Since the DNA chip and the kit according to the present invention include the above mentioned probes it can effectively identify species-specific SNPs according to the DNA hybridization without necessitating the analysis of the nucleotide sequences of a give diatom species thereby promptly determining its genotype and species. Further, only by a single test, a given diatom species can be identified of its genotype with accuracy and it can be contributed to the standardization and automation of the species identification process.

The present invention can be better understood by the examples described hereinbelow. While the invention will be described in conjunction with the exemplary embodiment, it will be understood that present description is not intended to limit the invention to the exemplary embodiment. On the contrary, the invention is intended to cover not only the exemplary embodiment, but also various alternatives, modifications, equivalents and other embodiments, which may be included within the spirit and scope of the invention as defined by the appended claims.

### EXAMPLES

### Example 1

### Synthesis of Primers

First, primers were synthesized for the amplification of DNAs extracted from diatoms. The diatoms and the primers used therefrom in the examples are the same as shown in Table 1.

Reverse (antisense) primers used in symmetrical or asymmetrical PCR reactions were synthesized with rhodamine, cy3 and cy5 attached at their terminal end or tagged with biotin so as to confirm the fluorescence after hybridization. They were synthesized so that they can bind to Syreptavidin-Cyanine after hybridization and used as such.

### Example 2

### DNA Extraction of Diatom Species and PCR Reaction

Thirty six major diatom species found in the coastal area of Korea, i.e., Achnanthes longipes, Chaetoceros atlanticus, Chaetoceros septentrionalis, Chaetoceros vistulae, Coscinodiscus rothii. Cylindrotheca fusiformis, Cymstosira lorenziana, Melosira numnuloides, Navicula sp., Nitzschia pungens, Nitzschia subpacifica, Skeletonema costatum, Stephanopyxis turris, Thalassiosira allenii, Thalassiosira baltica. Thalassiosira conferta, and Thalassiosira ostupii were extracted using DNesay tissue kit purchased from QIAGEN of Germany.

PCR reaction was performed using DNA Engine(MJ Research Inc., U.S.A.) under the conditions as shown in Table 3 below.

TABLE 3
Conditions for Confirming Normal Amplification of Primes

| Reaction Composition | | Reaction Condition | |
|---|---|---|---|
| Sterile distilled water | 9.5 gL | 94° C., 5 min | 1 cycle |
| 10X PCR buffer | 2 gL | 94° C., 30 sec. 49° C., | 40 cycle |
| 2.5 mM dNTP | 2 gL | 30 sec. 72° C., 1 min | |
| 10 gM forward | 1 gL | | |
| 10 gM reverse | 1 gL | | |
| 1 unit Hot start Taq | 0.5 gL | | |
| Purified DNA | 4 gL | 72° C., 7 min | 1 cycle |

The PCR products were electrophoresed in an agarose gel containing EtBr and confirmed by using Image Analyzer attached with UV transluminator.

### Example 3

### Synthesis of Probes for SEQ. ID. NOs.: 3˜51

In this Example, the probes for the respective 36 major diatom species were synthesized. The information on the nucleotide sequences of the probes for cross hybridization is shown in Table 1. Each species-specific probe for the respective diatom species was determined based on the nucleotide sequence domain with the lowest sequence homology by means of the multiple comparison of the nucleotide sequences of the COI gene of mitochondrial DNA of the diatom species.

First, on the top of a glass treated with aldehyde functional group, an amino link was modified to the 5′ terminus of a polynucleotide having the above nucleotide sequences, and an oligo (dT) nucleotide of 10-20 bp was added so as to minimize the spatial hindrance accumulated on the top of the glass among probes, and thereby completed the synthesis of the probes according to the present invention. The probes used in the present invention were synthesized in Metabion International AG, Germany.

### Example 4

### DNA Chip Synthesis

50 gM of a probe for the identification of diatom species where amino link prepared in Example 3 is modified on a slide was accumulated by mixing it with an equal amount of 3×SSC, and allowed to react at room temperature for 16 hours. Upon completion of the reaction, the slide was washed twice with 0.1% SDS for 5 minutes, reacted with sodium borohydride solution (1.3 g NaBH₄, 375 mL PBS, 125 mL EtOH) for minutes, washed with distilled water, dried by centrifuging at 800 rpm in a vacuum centrifuge for 10 minutes, and then stored at room temperature. The mimetic diagram of thus synthesized DNA chip structure is shown in FIG. 37.

FIG. 37 is a mimetic diagram of a DNA chip for the identification of diatom species according to an embodiment of the present invention which includes an oligonucleotide probe synthesized based on SNPs shown in the above FIGS. 1-36. As shown therein, the present invention is characterized in that each dot on a slide, which is used as a substrate for the manufacture of DNA chip, was accumulated such that it could include only one probe in each dot, whereas probes of a position marker were accumulated in a left upper dot and a right lower dot thereby utilizing them in the comparative analysis with the result of fluorescent scan.

### Example 5

### Hybridization Reaction

10 gL of the PCR product obtained in Example 2 was denatured at 99° C. for 3 minutes, mixed with hybridization solution. The mixture was coated on the DNA chip prepared in Example 4, and then allowed to react in a wet reactor for 1 hour. After hybridization, the resultant was washed with a mixed solution of 1×SSC and 0.1% sarcosyl for 5 minutes, washed with 1×SSC for 5 minutes, washed with 0.1×SSC for 1 minute while stirring, and then dried by centrifuging at 800 rpm in a vacuum centrifuge for 5 minutes.

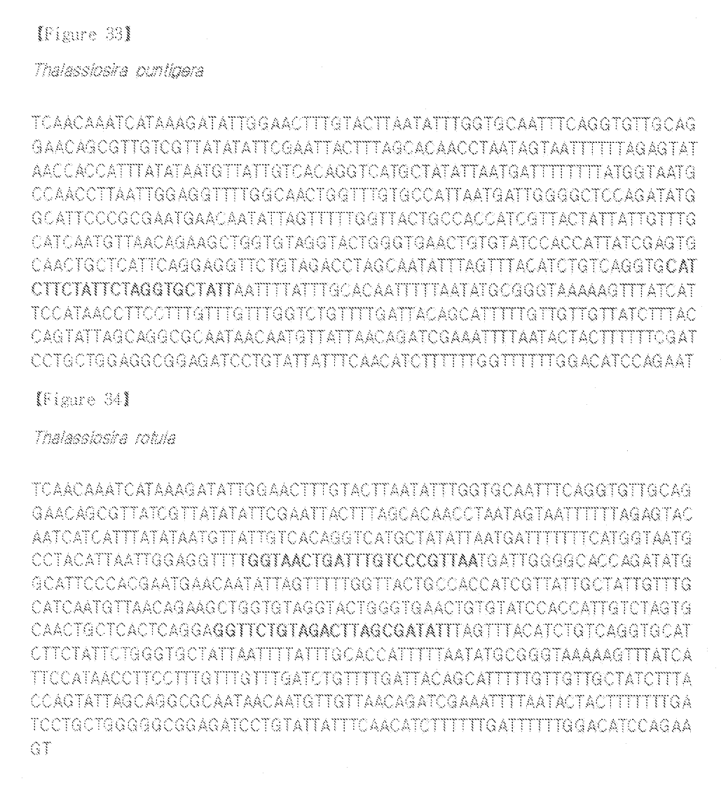

The results of the hybridization analyzed by using GenePix 4000B scanner (Molecular Device, U.S.A.) revealed that the distribution of fluorescence shown in DNA chips varied among diatom species thus confirming there is apparently a discernible difference among diatom species. The results are shown in FIGS. 38-54.

FIGS. 38-54 are pictures showing the hybridization reaction between oligonucleotide probes prepared according to the present invention and the amplified PCR products of specific diatom species including Achanthes longipes.

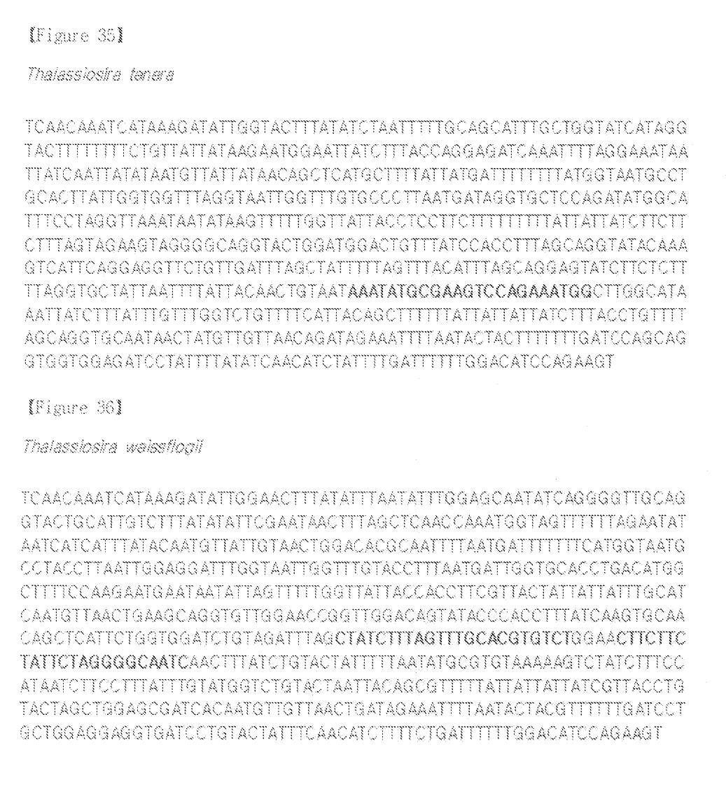

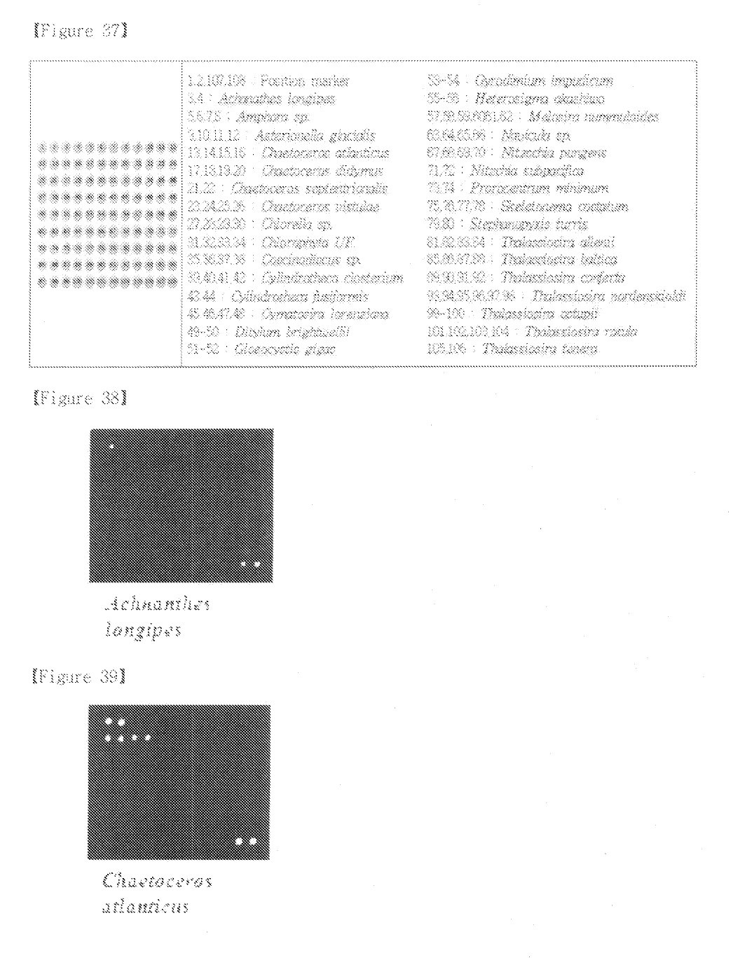

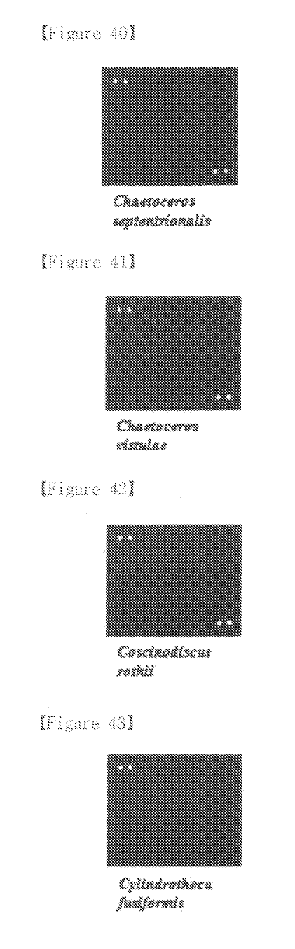

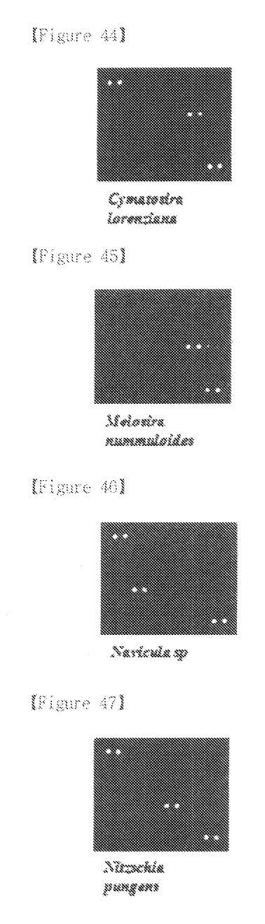

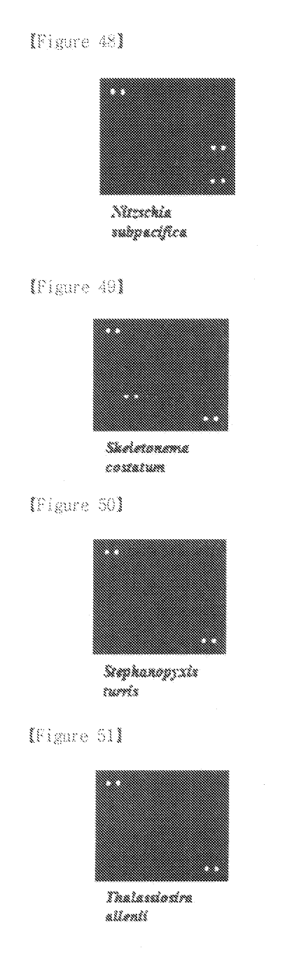

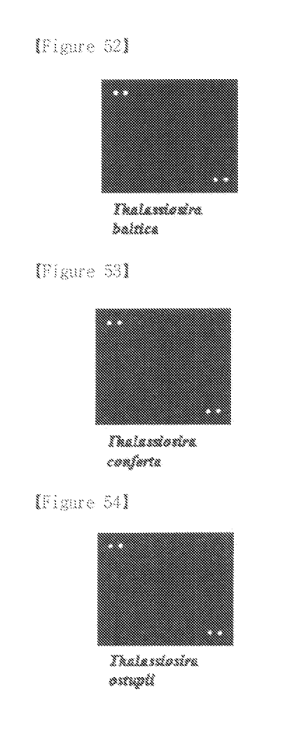

Herein, FIG. 38 shows the result of Achanthes longipes, FIG. 39 Chaetoceros atlanticus, FIG. 40 Chaetoceros septentrionalis, FIG. 41 Chaetoceros vistulae, FIG. 42 Coscinodiscus rothii. FIG. 43 Cylindrotheca fusiformis, FIG. 44 Cymstosira lorenziana, FIG. 45 Melosira numnuloides, FIG. 46 Navicula sp., FIG. 47 Nitzschia pungens, FIG. 48 Nitzschia subpacifica, FIG. 49 Skeletonema costatum, FIG. 50 Stephanopyxis turris, FIG. 51 Thalassiosira allenii, FIG. 52 Thalassiosira baltica. FIG. 53 Thalassiosira conferta, and FIG. 54 Thalassiosira ostupii, respectively.

As shown in the above, the present invention confirmed that fluorescent marker varied among diatom species, and it was also confirmed that a given diatom species can be identified based on this variation.

While an exemplary embodiment of the present invention has been described in detail, the protection scope of the present invention is not limited to the foregoing embodiment and it will be appreciated by those skilled in the art that various modifications and improvements using the basic concept of the present invention defined in the appended claims are also included in the protection scope of the present invention.

### INDUSTRIAL APPLICABILITY

The present invention has an advantage that it enables the identification of genotypes of various marine diatoms in a convenient, fast and accurate way by placing their specimens on a slide. By using the COI domains of mitochondria of 30 marine diatoms as genetic markers according to the present invention, it is possible to resolve the relatively low resolving power by the conventional method of identifying species based on morphological differences. The present method of identifying a given species based on SNPs is a more advanced method of genetic analysis than the conventional method enabling a more effective identification of a given species and improved resolving power.

Further, by the microarray method using a polynucleotide probe according to the present invention it is now possible to promptly identify the genotype of a given species based on the DNA hybridization result on species-specific SNPs without requiring sequence analysis of its gene. In addition, since an accurate analysis of genotypes is possible with a single test it has greatly increased the level of technology applicability via standardization and automation. That is, as compared with the conventional method, the present invention has considerably reduced the time required for the analysis and treatment of a large amount of specimens.

1. A method for identifying the diatom species, comprising:
obtaining a PCR product by performing a PCR reaction using DNA extracted from diatoms;
binding the PCR product respectively to a probe which is the same as or complementary to one or more of the nucleotide sequences selected from SEQ. ID. NOs. 3˜51; and
identifying the species of a given diatom based on the result of the above binding.

2. The method for identifying the diatom species according to claim 1, wherein the diatom species is limited to those having their habitat in the coastal area of Korea.

3. The method for identifying the diatom species according to claim 1, wherein the diatom species is at least one selected from the group consisting of Achnanthes longipes, Amphora sp., Asterionella glacialis, Chaetoceros atlanticus, Chaetoceros didymus, Chaetoceros septentrionalis, Chaetoceros vistulae, Chlorella ellipsoidea, C. schroeteri, Chlorophyta UF, Coscinodiscus perforatus, C. rothii. Cylindrotheca closterium, Cylindrotheca fusiformis, Cymstosira lorenziana, Ditylum brightwellii, Gloeocytis gigas, Gyrodimium impudicum, Heterosigma akashiwo, Melosira numnuloides, Navicula sp., Nitzschia pungens, Nitzschia subpacifica, Prorocentrum minimum, Skeletonema costatum, Stephanopyxis turris, Thalassiosira allenii, Thalassiosira baltica. T. decepiens, T. puntigera, Thalassiosira conferta, Thalassiosira nordenskioldi, T. weissflogii, Thalassiosira ostupii, and Thalassiosira rotula.

4. The method for identifying the diatom species according to claim 1, wherein the extracted DNA includes single nucleotide polymorphism (SNPs) domain of COI gene among mitochondrial DNAs of the diatom species.

5. The method for identifying the diatom species according to claim 1, wherein the probe binds to single nucleotide polymorphism (SNPs) domain of COI gene among mitochondrial DNAs of the diatom species, and the binding varies depending on the diatom species.

6. The method for identifying the diatom species according to claim 5, wherein the binding differs from diatom to diatom such that the probe including SEQ. ID. NO.: 3 binds specifically to 26^{th}-49^{th} DNA sequence of COI gene of mitochondria DNA of Achnanthes longipes, the probe including SEQ. ID. NO.: 4 to 544^{th}-577^{th} DNA sequence of Amphora sp., the probe including SEQ. ID. NO.: 5 to 55^{th}-78^{th} DNA sequence of Amphora sp., the probe including SEQ. ID. NO.: 6 to 706^{th}-729^{th} DNA sequence of Asterionella glacialis, the probe including SEQ. ID. NO.: 7 to 485^{th}-508^{th} DNA sequence of Asterionella glacialis, the probe including SEQ. ID. NO.: 8 to 58^{th}-81^{st} DNA sequence of Chaetoceros atlanticus, the probe including SEQ. ID. NO.: 9 to 39^{th}-62^{nd} DNA sequence of Chaetoceros atlanticus, the probe including SEQ. ID. NO.: 10 to 638^{th}-706^{th} DNA sequence of Chaetoceros didymus, the probe including SEQ. ID. NO.: 11 to 625^{th}-648^{th} DNA sequence of Chaetoceros didymus, the probe including SEQ. ID. NO.: 12 to 639^{th}-662^{nd} DNA sequence of Chaetoceros septentrionalis, the probe including SEQ. ID. NO.: 13 to 585^{th}-608^{th} DNA sequence of Chaetoceros vistulae, the probe including SEQ. ID. NO.: 14 to 447^{th}-470^{th} DNA sequence of Chaetoceros vistulae, the probe including SEQ. ID. NO.: 15 to 706^{th}-729^{th} DNA sequence of Chlorella ellipsoidea and C. schroeteri, the probe including SEQ. ID. NO.: 16 to 551^{st}-574^{th} DNA sequence of Chlorella ellipsoidea and C. schroeteri, the probe including SEQ. ID. NO.: 17 to 709^{th}-730^{th} DNA sequence of Chlorophyta UF, the probe including SEQ. ID. NO.: 18 to 654^{th}-675^{th} DNA sequence of Chlorophyta UF, the probe including SEQ. ID. NO.: 19 to 635^{th}-658^{th} DNA sequence of Coscinodiscus perforatus and C. rothii., the probe including SEQ. ID. NO.: 20 to 482^{nd}-505^{th} DNA sequence of Coscinodiscus perforatus and C. rothii., the probe including SEQ. ID. NO.: 21 to 547^{th}-570^{th} DNA sequence of Cylindrotheca closterium, the probe including SEQ. ID. NO.: 22 to 534^{th}-557^{th} DNA sequence of Cylindrotheca closterium, the probe including SEQ. ID. NO.: 23 to 700^{th}-723^{th} DNA sequence of Cylindrotheca fusiformi, the probe including SEQ. ID. NO.: 24 to 684^{th}-704^{th} DNA sequence of Cymstosira lorenziana, the probe including SEQ. ID. NO.: 25 to 651^{st}-674^{th} DNA sequence of Cymstosira lorenziana, the probe including SEQ. ID. NO.: 26 to 478^{th}-501^{st} DNA sequence of Ditylum brightwellii, the probe including SEQ. ID. NO.: 27 to 540^{th}-563^{th} DNA sequence of Gloeocytis gigas, the probe including SEQ. ID. NO.: 28 to 677^{th}-700^{th} DNA sequence of Gyrodimium impudicum, the probe including SEQ. ID. NO.: 29 to 580^{th}-601^{st} DNA sequence of Heterosigma akashiwo, the probe including SEQ. ID. NO.: 30 to 707^{th}-729^{th} DNA sequence of Melosira numnuloides, the probe including SEQ. ID. NO.: 31 to 681^{st}-704^{th} DNA sequence of Melosira numnuloides, the probe including SEQ. ID. NO.: 32 to 626^{th}-649^{th} DNA sequence of Melosira numnuloides, the probe including SEQ. ID. NO.: 33 to 706^{th}-729^{th} DNA sequence of Navicula sp., the probe including SEQ. ID. NO.: 34 to 580^{th}-603^{th} DNA sequence of Navicula sp., the probe including SEQ. ID. NO.: 35 to 100^{th}-723^{rd} DNA sequence of Nitzschia pungens, the probe including SEQ. ID. NO.: 36 to 634^{th}-657^{th} DNA sequence of Nitzschia pungens, the probe including SEQ. ID. NO.: 37 to 710^{th}-733^{rd} DNA sequence of Nitzschia subpacifia, the probe including SEQ. ID. NO.: 38 to 21^{st}-44^{th} DNA sequence of Prorocentrum minimum, the probe including SEQ. ID. NO.: 39 to 712^{th}-734^{th} DNA sequence of Skeletonema costatum, the probe including SEQ. ID. NO.: 40 to 626^{th}-649^{th} DNA sequence of Skeletonema costatum, the probe including SEQ. ID. NO.: 41 to 571^{st}-594^{th} DNA sequence of Stephanopyxis turris, the probe including SEQ. ID. NO.: 42 to 678^{th}-699^{th} DNA sequence of Thalassiosira allenii, the probe including SEQ. ID. NO.: 43 to 651^{st}-674^{th} DNA sequence of Thalassiosira allenii, the probe including SEQ. ID. NO.: 44 to 491^{st}-514^{th} DNA sequence of Thalassiosira baltica. T. decepiens and T. puntigera, the probe including SEQ. ID. NO.: 45 to 637^{st}-660^{th} DNA sequence of Thalassiosira conferta, the probe including SEQ. ID. NO.: 46 to 491^{st}-514^{th} DNA sequence of Thalassiosira nordenskioldi and T. weissflogii, the probe including SEQ. ID. NO.: 47 to 464^{th}-487^{th} DNA sequence of Thalassiosira nordenskioldi and T. weissflogii, the probe including SEQ. ID. NO.: 48 to 491^{st}-514^{th} DNA sequence of Thalassiosira nordenskioldi and T. weissflogii, the probe including SEQ. ID. NO.: 49 to 584^{st}-607^{th} DNA sequence of Thalassiosira ostupii, the probe including SEQ. ID. NO.: 50 to 242^{nd}-265^{th} DNA sequence of Thalassiosira rotula, and the probe including SEQ. ID. NO.: 51 to 448^{th}-471^{th} DNA sequence of Thalassiosira rotula.

7. The method for identifying the diatom species according to claim 1, wherein the step of binding the PCT product to the probe is conducted at least two times.

8. The method for identifying the diatom species according to claim 1, wherein the PCR reaction is performed by using the polynucleotide, which respectively includes a nucleotide sequence being the same as or complementary to that of SEQ. ID. NO: 1 or SEQ. ID. NO: 2, as a forward primer or a reverse primer.

9. A probe for identifying the diatom species consisting of one or more of polynucleotide, which respectively includes a nucleotide sequence being the same as or complementary to one or more DNA selected from SEQ. ID. NOs: 3˜51.

10. A DNA chip for identifying the diatom species consisting of one or more of polynucleotide, which respectively includes a nucleotide sequence being the same as or complementary to one or more DNA selected from SEQ. ID. NOs: 3˜51.

11. The DNA chip for identifying the diatom species according to claim 10, further comprising a position marker.

12. A kit for identifying the diatom species including polynucleotide probes having 15-30 bp long continuous nucleotide sequences being the same as or complementary to one or more DNAs selected from SEQ. ID. NOs. 3˜51, which bind to the single nucleotide polymorphism (SNPs) domain of COI gene among mitochondrial DNAs of diatom species;
and a primer for PCR amplification.

13. The kit for identifying the diatom species according to claim 12, wherein the primer is a forward primer or a reverse primer, which respectively includes a nucleotide sequence being the same as or complementary to that of SEQ. ID. NO: 1 or SEQ. ID. NO: 2.


SEQUENCE LISTING (36 sequences)

SEQ ID NO: 1 - DNA, 746 bp, Achnanthes longipes
tcaacaaatcataaagatattggcacgctctatctgatttttgcagtgattgcgggcatcatcggcggcgcgatgtctggtgttatgcgcgaagagcttgcgcagcctggcatgcagattttaacgaactttaccgatggcaacttggattcagcgtaccatctttggaacgtgttcatcacagcgcacggcctaatcatgatcttcttcatggtgatgcccgcgatgattggtggctttggcaactggtttgtgccgatcatgattggcgcgccggatatggcgtttccgcggatgaacaacatttctttctggctgcttccgccagcgttcttcttactgctgttcagcgtgtttctggaaggccctccaggccaaaacggcgttggcggcggctggacgatctatcccccgctcagcacatccggccagcctgggccagcgatggatatggcgattttctccctgcatattgcgggcgccagctctatcctcggggcgattaatttcatcaccaccatttttaacatgcgcgcgcctggcatgacgctgtttaaaatgccgctgtttgtatggtctgtattgatcacagcgttcttgctgctgctgagcctgcccgtactcgcgggcgcaattaccatgctgttgacagaccggaactttggcacgtccttctttgacgcctcagcaggcggggaccctgtgctcttccagcacttgttctgattttttggacatccagaagt

SEQ ID NO: 2 - DNA, 710 bp, Amphora sp.
tcaacaaatcataaagatattggtactttatatatatttttaggagcatttgcaggtgttattggaactgtagtttctgtaatgatacgtacagaattaggtggaattggtgatcaaattttacaaggaaattatcaattttataatgttttaattactgctcacgcttttttaatgattttttttatggttatgcctattttaatgggaggttttggtaactgatttgtacctattatgattggtgcaccagatatggcatttccgagattaaataacattagtttttgactattaccaccatctcttttattattattaagctcttcttttgtagaaactggagcaggtactggttgaactgtttacccaccattaagtagtattcaagcacattctggaccatctgttgatttagccatttttagtttacacttatctggtttatcttctattttaggttctgtaaattttatcgtaactatttttaatatgagagctccaggcttttttatgcataaaattgaattatttgtttgagctgttttaattactgcatttttattattaatttctttaccagttttagcaggtgctattacaatgttattaactgatagaaattttaatactactttttttgatccagctggaggaggtgatcctgttttatatcaacatttattttgattttttggacatccagaagt

SEQ ID NO: 3 - DNA, 710 bp, Asterionella glacialis
tcaacaaatcataaagatattggaactttatatttaatatttggagcaatttctggggtagcaggcactgctttatcactatacattcgtttaacgttggcacatccaaacggcgattttttagcatataatcatcacttatataacgttatagttacaggacacgcatttgtaatgatttttttcatggtaatgccaacactaataggtggttttggtaactggttcgtaccattgatgattggagcacctgatatgtgtttcccacgtatgaacaatattagtttttggttattaccaccatctttattcttgttaatcgcttcggttctaactgaagcaagtgccggtaccggttggactgtttacccacctttatcaagtattacagcacactcaggtggttctgttgatttagcaatttttagtttacatttatctggtgcttcatcaatattaggtgctatcaattttatttgtacaattttcaatatgcgtgtaaaaagtttatcttttcataaactaccattatttgtttggtcagttttaattaccgcgtttttattactattatcgttaccagttttagcaggagcaatcacaatgttattaacagatagaaactttaatacaactttttttgacccagcaggtggaggcgatcctgtcttataccagcatttattctgattttttggacatccagaagt

SEQ ID NO: 4 - DNA, 710 bp, Chaetoceros atlanticus
tcaacaaatcataaagatattggtactttatatcttatttttggtgcaatatctggagtagctgggactgctctatctttatatattcgaattactttagcacagcctaacagtagtttcttagagcataatcatcaaatgtataatgttattgtaacaggacatgcttttgttatgatttttttcatggttatgccaactttaattggaggttttggtaactggttcgttccgttaatgatcggtgcacctgatatggcatttccacgaatgaataatattagtttttggttattaccaccttcattgttattattgattgcttcaattttagctgaagcaggagtaggtacaggttggactgtttaccctcctttatctagcggtacatcacactcaggtggagctgtagatttagctatttttagtttacacttatctggggcttcatcaattttaggagctattaactttatttgtactatttttaatatgagagttaaaagtttatcatttcataaattacctttatttgtatgggcagtgttaattacagcatttttacttttattatcactaccagtattagcaggtgctattacaatgttattaactgatagaaatttcaatacaaccttttttgatccagcaggaggaggtgacccaattttataccaacatttattttgattttttggacatccagaagt

SEQ ID NO: 5 - DNA, 710 bp, Chaetoceros didymus
tcaacaaatcataaagatattggaacactgtatttaatttttggggctatttcaggagtggctggaacagctctttcgttatatatcagaataactttagcacaaccaaacagtacttttttagaacaaaattttcaaatgtataatgttattgtaaccggtcacgcttttgttatgattttttttatggtaatgcctactttaataggaggatttggtaattggttcgttccattaatgattggagcacctgatatggcatttccaagaatgaacaatataagtttttggttactaccaccgtcacttttactattaattgcttctattttaacagaagcaggtgtaggaacaggctggactgtttatccccctttatctagcggaacctctcactcaggaggggctgtagatctagctatttttagtttacacttatcaggggcatcttctattttaggtgcaataaattttatttgcactatttttaacatgagagttaaaagtttatcttttcataagctacctttatttgtttgggcagttttaattacagcctttttattattattatctttaccagtgcttgcgggagctattactatgttattaacagatagaaacttcaacactactttttttgaccctgcagggggtggagatcctgtgttgtatcaacatctattctggttttttggacatccagaagt

SEQ ID NO: 6 - DNA, 710 bp, Chaetoceros septentrionalis
tcaacaaatcataaagatattggaactttatatttaattttcggagcgatttctggtgtaatgggaacagctttgtctttatacataagattgactttataccaaccaaatggtagttttttagagaacaaccatcatttgtacaatgttgtggttactggacatgcatttgttatgatcttttttatggtaatgccaacattaattggcggatttggtaattggtttgtacctttaatgataggtgcacctgatatggcttttcctagaatgaataacattagtttttggttattaccaccatctttattaatgttattcgcttcgattttatctgaagcaggtgtaggtactggttggactgtttatcctcctctatcaagtggtggatctcattctggtggcgctgttgatttagcgatttttagtttacatttgtcgggagcttcttcaattttaggagctattaattttattacaacaatttttaatatgcgagtaaaaagtttatattttcataatttacctttattcgtatggtcagttttaattacagcttttttattgcttttatctttacctgttttagctggggcaattaccatgcttttaactgatcgaaatttcaatactacctttttcgatccagctggtggtggcgatcctgtattgtatcaacatttattttgattttttggacatccagaagt

SEQ ID NO: 7 - DNA, 710 bp, Chaetoceros vistulae
tcaacaaatcataaagatattggaacgttataccttatttttggcgcaatttctggcgtggctggtactgctttatctttatatattcgattaacattagctcaaccaaatggtggcttcctagaatacaatcaccaaatgtacaacgtaatcgttacaggacacgcttttgtaatgattttttttatggttatgccaactttaattggaggttttggtaactggttcgttcccttaatgatcggagcgcctgatatggcttttccacggatgaacaatataagtttttggttactaccgccatcattgttattattagttgcttctattctttcagaagcaggggtaggtactggttggactgtatatccaccattatctagcggaacctcgcattcaggtggcgcagtggatttagctatttttagtctacatttgtctggagcttcttctattttaggtgctataaactttatttgtactatatttaatatgcgagtaaaaagtttatcttttcataaattacctttattcgtttgggctgtcttaataacagctttcttgttattgttatcgttacctgtactagctggagctattacaatgttgttaactgatagaaattttaacactacattctttgatccggcaggtggcggtgatcctgtattgtatcaacatttattttgattttttggacatccagaagt

SEQ ID NO: 8 - DNA, 710 bp, Chlorella ellipsoidea
tcaacaaatcataaagatattggaaccctttatttaatttttggagctattgctggagttgctggaacaactctttctgttctaattcgattagaattagctcaaccaggaaatcagtttttatctggaaataatcagttatataacgttattgtaacaggacacgcgttcgttatgatttttttttttgtgatgcctgttcttattggcggttttggtaactggtttgtacctttaatgattggtgctcctgatatggctttcccacgaatgaataacattagtttttggttattacctccttctcttattcttttattggcttcaacctttgttgaagctggtgcgggaactggttggaccgtgtatccccctttaagtggcgctcaagctcactcaggtccttccgtggatttagctatatttagtcttcacctttcaggtgctgcttcaattttaggtgctattaactttattactactatttttaatatgcgtgcacctggtatgagtatgcatagactacctttatttgtgtggtctgttttaattactgctttcttgcttttattgtcgcttcctgtttttgctggagcaattactatgttattgactgatagaaactttaatactactttttacgatcctgcaggaggaggtgatcccgtattgtaccaacatttattctgattttttggacatccagaagt

SEQ ID NO: 9 - DNA, 710 bp, Chlorella schroeteri; identical to SEQ ID NO: 8

SEQ ID NO: 10 - DNA, 698 bp, Chlorophyta UF
tcaacaaatcataaagatattggaaccatgtacttgtggttttcttttgcaatgttcattttgggcggtgcattcgcaatgatcattcgtgccgagctgttccagcccggtatgcaattgatcgagcccgcattctttaaccaaatgacaaccttgcacggcttgattatggtcttcggtgccatcatgccgtcttttgtgggcttggctaactggatgattccgatgatgatcggtgcgccagacatggcattgccgcgtatgaacaattggtcgttttggttgttgccccccgcctttttgattttggcagggacgctgttcatggaaggtggagcgcccgcatttggttggactttttatgcgcctctgtcgaccacgtatgcaccgccctcggtcacttatttcatcttctcaatccacgtgctaggcatgtcctctattatgggagcgattaacatcatcgcgaccattatgaacatgcgcgcgcctggtatgacctacatgaaaatgccactgttcgtttggacatggttgattaccgcatttttgcttgttgccgtgatgccggttttggcgggcgcagtcactatgatgctgatggacatccacttcggcaccagcttcttctcagctgcgggtggtggtgacccagtcttgttccagcacattttctgattttttggacatccagaagt

SEQ ID NO: 11 - DNA, 710 bp, Coscinodiscus rothii
tcaacaaatcataaagatattggtaccttatatttaatttttggagcattttcaggggttgcaggtacgactctttcaatgtttataagattaaatttatcgttacctaacggtcaatttttagacaataattatcaattatacaatgttatagttactggtcatgcttttgtaatgattttttttatggttatgccagttttaattggaggttttggtaattggtttgtgccattaatgataggagcacctgatatggcttttcctagaatgaataatatcagtttttggttattacctccttcactagttttattgactatatctatgttagcagaagcaggggctggtactggatggactgtttacccaccattatctagtgtaaatgcacattcaggtgcttctgtagatttagctatttttagccttcatttatcgggagctgcttctattttaggtgctataaattttatatgtactattttaaatatgagagtaaaaggattgtttatgcatagattacctctatttgtttggtctattttaattactgcagtattattattgttatcgttgccggtattagcaggtgctatcacaatgcttttaactgatagaaactttaatactactttttttgatccagctggaggaggtgatcctgtattatttcaacatttattctggttttttggacatccagaagt

SEQ ID NO: 12 - DNA, 709 bp, Coscinodiscus perforatus
tcaacaaatcataaagatattggtaccttatatttaatttttggagcattttcaggggttgcaggtacgactctttcaatgtttataagattaaatttatcgttacctaacggtcaatttttagacaataattatcaattatacaatgttatagttactggtcatgcttttgtaatgattttttttatggttatgccagttttaattggaggttttggtaattggtttgtgccattaatgataggagcacctgatatggcttttcctagaatgaataatatcagtttttggttattacctccttcactagttttattgactatatctatgttagcagaagcaggggctggtactggatggactgtttaccaccattatctagtgtaaatgcacattcaggtgcttctgtagatttagctatttttagccttcatttatcgggagctgcttctattttaggtgctataaattttatatgtactattttaaatatgagagtaaaaggattgtttatgcatagattacctctatttgtttggtctattttaattactgcagtattattattgttatcgttgccggtattagcaggtgctatcacaatgcttttaactgatagaaactttaatactactttttttgatccagctggaggaggtgatcctgtattatttcaacatttattctgattttttggacatccagaagt

SEQ ID NO: 13 - DNA, 710 bp, Cylindrotheca closterium
tcaacaaatcataaagatattggtactttatatctaatttttgcagcatttgctggtatcataggtacttttttttctgttattataagaatggaattatctttaccaggagatcaaattttaggaaataattatcaattatataatgttattataacagctcatgcttttattatgattttttttatggtaatgcctgcacttattggtggtttaggtaattggtttgtgcctttaatgataggtgctccagatatggcctttcctaggttaaataatataagtttttggttattacctccttctttttttttattattatcttcttctttagtggaagtaggggcaggtactggatggactgtttatccacctttagcaggtatacaaagtcattcaggaggttctgttgatttagctatttttagtttacatttagcaggagtatcttctcttttaggtgctattaattttattacaactgtaataaatatgcgaagtccagaaatggcttggcataaattatctttatttgtttggtctgtttttattacagcttttttattattattatctttacctgttttagcaggtgcaataactacgttgttaacagatagaaattttaatactactttttttgatccagcaggtggtggagatcctattttatatcaacatttattttgattttttggacatccagaagt

SEQ ID NO: 14 - DNA, 710 bp, Cylindrotheca fusiformis
tcaacaaatcataaagatattggtacattataccttatttttgctttatttgcaggagtaataggtacggtattttctatatttattcgtatggaattagcgacaacaggtgatcaaatttttaatggaaattatcagttatataatgtagttattactgcgcatgcttttattatgattttttttatggtaatgcctgcgttaataggtggttttggtaattggtttgttcctttaatgttaggtgctcccgatatggccttccctagattaaataatattagtttttggttattaccaccatctttcttattattattatcttcttctttagtagaggtaggggctggtactggttgaactgtttatccgccattatctggtatagctgcgcattcaggaggttctgttgatttagctatttttagtctacatttagcaggagtttcttcattattaggtgctattaattttataacaacaatatttaatatgagagcaaataattttagtatttataaaatgccattatttgtttgagcagtattaattacagcatttttattattattatcattacctgttttagctggtgcaattactatgttattaactgatcgtaattttaatactactttttttgatcctgcaggaggaggtgatccaatactttatcaacatctattttgattttttggacatccagaagt

SEQ ID NO: 15 - DNA, 695 bp, Cymatosira lorenziana
tcaacaaatcataaagatattggcacactgtatttagtatttgcactagtgatgtttatggttggtggtgcaatggcaatggttatacgcttggaattatttcaaccaggtttacaatttattgatccaggcttctttaatcaaatgacaactgttcatgctttagtcatgatttttggtgctgttatgccggcttttgtgggtttagccaattggatgttgcccattatgattggcggccctgatatggcattacctaggatgaataactggagtttttggatactaccttttgcatttacaatgctattggcaacattctttatggatggtggtgcgccagcaggtggctggactatgtacccaccgttagtactacaaggtggtaacggctttccatttatgatttttgcgattcatatgatgggtatttcatctgtgatgggcgctataaacgttattgttaccatccttaatatgcgtgctcctggcatgacattgatgaaaatgcctttatttgtatggacttggttcattacagcttatttattaattgccgtaatgcctgtgttagcaggggcaatcacaatgcttttgacagatcgtttttacgatacaacgttctttagtgcaacgggtggtggtgatcctgtgttgttccagcatattttctggttttttggacatccagaagt

SEQ ID NO: 16 - DNA, 708 bp, Ditylum brightwellii
tcaacaaatcataaagatattggtactttatatttaatctttggtgctatatctggtgttgctggtacagctctatcattatatattagattaactttatatcaaccaaatagtggttttttagaaaataaccatcatttatataatgttattgttactggtcatgcttttgttatgatttttttatggtaatgcccactttaattagtggttttggtaactggtttgttcctttaatgataggagctcctgatatggcatttcctagaatgaataatataagtttttggttattacctccttctttattacttttatttgcttctattttagctgaatcaggggcaggcactggttggactgtttacccaccattatcaagtgctacagcacactcaggaggtgctgtagatttagctattttcagtttacatctttctggtgcttcttctattttaggtgctattaattttatttgtactatttttaatatgcgtgtaaaaagtttatcttttcataatttacctctatttgtatggtcagttttaataacagcatttttattattattatcattaccagtattagctggtgcaataactatgcttttaactgatagaaattttaatactactttttttgatcctgcgggtggtggtgaccctgttttatatcaacatttattttgattttttggacatccagaag

SEQ ID NO: 17 - DNA, 710 bp, Gloeocystis gigas
tcaacaaatcataaagatattggtaccttatatattattttaggaggtattgctggtatttttggtacattattatctattttaattagaatggaattagctgctccaggtaatcaaatttttaatggtaatcatcaagcgtataatgttgtagttattgcacatgcatttgtaatgattttttttatggttatgcccattttaataggcggttatggaaattggtttgttcctttattaataggagctcctgatatggcttttcctcgtttaaataatataagtttttggtttttaccaccatcatttatattattattaaattctgcattagttgaggctggtgcaggtactggatggactgtttatccacctttaagtagtgctttagcccattcaggagcttctgttgatctagctatttttagtttacatttagcaggggtttcatctttattaggggctattaattttattgttactataattaatatgagagcaataggaatgacatttcatagattacctctttttgtttgggctgtttttgttactgtatttttattattaatatcattaccagtattagcaggggctattactatgttattaaccgatcgaaattttaatacaatgttttttgatccagcaggtggaggagatcctattttatatcaacatttattttggttttttggacatccagaagt

SEQ ID NO: 18 - DNA, 709 bp, Gyrodimium impudicum
tcacaaatcataaagatattggaacactttatttaatttttggtggtattgccggagttatgggaaccactatgtcaattttaattcgaatggaattagcttatcctggaagtcaaattttagcaggtaaccatcaactttataacgttctagtgactgggcacgcttttgtgatgatcttcttcatggtaatgccagttttaattggtggttttggaaattggtttgttcctttaatgattggagcaccagacatggctttccctcgaatgaacaatattagtttttggttattaccgccatcgttattattgttattagcgtctactctagtagaagcaggagcaggaaccggttggactgtgtacccaccgttaagtagcgctcaagctcactcaggaccgtcggtagatttagctattttcagtttacacgtttcaggagcagcatcaattttaggggcaattaattttattaccactattttaaacatgcgagcacctggtatgaccatgcatcgactaccgttgtttgtgtgggctgtgtttattactgcaattttattattattatcgttaccagtattagcaggagcaattactatgttattaactgatcgaaatttcaacactaccttttacgatccggcaggaggaggagacccagtattgtatcaacatttattttgattttttggacatccagaagt

SEQ ID NO: 19 - DNA, 710 bp, Heterosigma akashiwo
tcaacaaatcataaagatattggtactttatatttaatttttggtgctatttctggtttagttgggcttattttctcaatggctattcgtcttgagctttctcaaccaggaaatgctttcttagaaggtaatcaccaactttacaatgtgttagtaactgctcacgcatttatcatgatcttttttatggttatgccaagcctacttggtggttttggtaactggatgattcctattatgataggagctcctgatatgtctttccctcgtttaaacaatataagtttctggcttttaccaccagctttaattttactatttgcttcatctttagttgaagtaggtgctggtactggttggactgtttaccctcctctaagtggaattcaagctcattcaggaggttctgttgatttagctattttctcgcttcacttagctggagtttcatctattttaggggctatcaactttatcacaactatatttaacatgcgtactcctgggatgactgctcatcgtctagggttatttccttgggctatccttatcacagcttttttactacttttatcattacctgttctagcaggtgctattactatgcttcttactgaccgtaactttaatacaacgttcttcgacccagccggtggtggtgatgttttactttatcaacatcttttttggttttttggacatccagaagt

SEQ ID NO: 20 - DNA, 710 bp, Melosira nummuloides
tcaacaaatcataaagatattggtacgttgtacttgctttttggagcattttcaggagtagcagggactactctttcactattcataagattaactttagaatctcctgcaagtaattttttaagtggtaactaccaattgtacaacgttattgttactggtcacgcttttcttatgatttttttcatggtaatgcctactctaattggaggatttgggaactggtttgttccaataatgataggtgctccagacatggcttttcccagaatgaataatattagtttttggttgttacctccctcactactgcttttaatttcttctgttctagcagaagccggagtaggtactggctggacagtctatcctcctctttctagcgggaattcgcattcaggccctgcagtagatcttgctatatttagtttacatttgtctggagccgcttctattttaggagcaattaatttcatatgcactattttaaacatgagaactaaaggtttatatatgcataaattgcccctttttgcttggtcaattttaataacagcagttttgcttttattgtctttacctgttcttgctggagctattactatgcttctaacggatagaaattttaatacaactttttttgaccccgcaggaggcggagatcccgttctttatcaacatctattttgattttttggacatccagaagt

SEQ ID NO: 21 - DNA, 710 bp, Navicula sp.
tcaacaaatcataaagatattggaactttatatattatttttggtggtatcgctggggtagctggtactgcattatctttatacataagaataactttatctcaacctaacggtagttttttagaatacaatcaccatttatacaacgtgattgtaacaggtcatgcgtttgttatgattttttttatggtaatgccaattttaattggtggttttgggaattggtttgttcctttaatgattggtgctcctgatatggcttttccccgaatgaataatattagtttttggttattacccccatctcttttattattaatcgagtctgttctttgtgaagctggggttggtactggttggactgtttacccaccattatctggtattattgctcattctggtggtgctgttgatttagcaattttcagtttacacctttctggtgctgcctctattttaggggcaattaatttcatatgtacaattgtgaatatgagaaccgaaagtcttccatttcacaaattacctttatttgtgtggtcagtatttttaacagcaattcttttattactatctttacctgtattagctggtgcaatcacaatgttattgaccgatagaaatttcaatacaacattttttgatccagccggtgggggtgatccagttttataccagcacttattttgattttttggacatccagaagt

SEQ ID NO: 22 - DNA, 700 bp, Nitzschia pungens
tcaacaaatcataaagatattggtactttatatttaatttttgcgtttattgcaggtgtaattggtactcttttttctattattattagattagaattggcttatcctggtgatcaaatattgaatggtaactatcaattttatatgtaattataactgcacatgcttttataatgattttttttatggttatgcctgctatgattggtggtttcggtattgatttttacctttaatgattggatctgctgatatggcttttccacgtttgaataatttaagtctttgattattgcctccatcatttacactattactattatcaagtattacaggtgcaggtgcaggaactggttgaacagtttatcctccattatcagcaatgacttctgattgttctgttgatttagcaatatttagtttacatttatcgggtatttcatctattttaggagctattaattttatagctacagttattatttaagattacctggttttaaattaagtgcattgcctttatttgtatgatctgttttaataacagcagtattgttattattatctttaccagtattagcagcaggtataactatgttgttaactgatagaaattttaatagttcattttttatccatctggaggaggagaccctatattatatcaacatttattttgattttttggacatccagaagt

SEQ ID NO: 23 - DNA, 710 bp, Nitzschia subpacifica
tcaacaaatcataaagatattggaaccttatatattatctttggagcaatttctggaatcgcaggaactgctttatcattatacattcgaattacactatctcaaccaaacaattcatttttagagtacaatcatcatttctacaacgtgattgttacaggtcacgctattctaatgatcttttttatggtaatgccaattttaatcggcgggttcgggaattggttcgtgccgctaatgattggtgcgcctgatatggcttttccaagaatgaataacattagtttttggttactacctccatctttactattactgattgaatcagttttatgtgaagcaggtgttggtactggttggacagtttaccctccactatcgggtgttatagctcactcaggaggttctgtagacctagcaattttcagtcttcatttatctggagctgcatctattttaggtgcaattaatttcatctgtactatcgtaaacatgcgaacagaaagtttacctttccataagttgcctctgtttgtgtgggctgttttcattactgctattttactgctattatctttaccggtattagcaggagcaattacaatgttacttacagatagaaattttaatactactttctttgacccagcgggtggtggagaccctgtgttattccagcacttattctgattttttggacatccagaagt

SEQ ID NO: 24 - DNA, 746 bp, Prorocentrum minimum
tcaacaaatcataaagatattggcacgctttatctcatttttgcagtgattgcgggcatcatcggcggcgcgatgtctggtgttatgcgcgaagagcttgcgcagcctggcatgcagattttaacgaactttaccgatggcaacttggattcagcgtaccatctttggaacgtgttcatcacggcgcacggcctgatcatgatcttcttcatggtgatgcccgcgatgattggtggctttggcaactggtttgtgccgatcatgattggcgcgccggatatggcgtttccgcggatgaacaacatttctttctggctgctcccgccagcgttcttcctgctgctgttcagcgtgtttcttgaaggccctccaggccaaaacggcgttggcggcggctggacgatctatcccccgctcagcacatccggccagcctgggccagcgatggatatggcgattttctccctgcatattgcgggcgccagctctatcctcggggcgattaatttcatcaccaccatttttaacatgcgcgcgcctggcatgacgctgtttaaaatgccgctgtttgtatggtctgtattgatcacagcgttcttgctgctgctgagcttgcctgtactcgcgggcgcaattaccatgctgctgacagaccggaactttggcacgtccttctttgacgcctcagcaggcggggaccctgtgctcttccagcacttgttctgattttttggacatccagaagt

SEQ ID NO: 25 - DNA, 710 bp, Skeletonema costatum
tcaacaaatcataaagatattggaactttatatttgatttttggagcaatatcaggtgttgctggaaccgcattgtctttatatattcgaatcactttagcccagccaaatagcagttttttagaatataatcaccatttatacaatgttattgtcacaggacatgctatacttatgatttttttcatggtaatgccaacattaattggaggatttggtaattggtttgttcccttaatgattggtgcgccagatatggctttcccacgaatgaataatattagtttttgattattgcctccttcattactgttattgtttgcatctatgttaactgaagcgggtgtaggtactggatgaaccatttacccacccttatcaagtgcaacagctcattctggaggttctgtagatttagcaatattcagtttacatttatcaggtgcgtcttctattttaggtgctattaattttatttgtactatcttcaatatgcgagtaaaaagtttatcttttcataatcttcctttatttgtatggtctgttttaataacagcatttttattattattatctctgcctgttttagctggagctattacaatgttattaactgatagaaattttaacactaccttttttgaccctgctggtggaggcgaccctgtcttgtttcagcatcttttctgattttttggacatccagaagt

SEQ ID NO: 26 - DNA, 710 bp, Stephanopyxis turris
tcaacaaatcataaagatattggtacattatatttaatttttggggcgttttctggtattgccggtaccacactttcattatttatacgccttaccttagaatcgcctggaaatgatatattaagtaacaatcatcaattatataatgttatagtaacaggtcatgctttcattatgattttttttatggtaatgcctacgttaatcggtggatttggtaattggtttgtacctataatgattggagctccagatatggcttttcctagaatgaataatataagtttttggttactacctccgtctttattgttattagtttcatctgtattatcagaagcaggagttggtacaggttggactgtataccctccgttgtctagtggtaattctcattcaggccctgctgtagatttagctatatttagtttacacttatcaggagcttcttctattttaggagcaattaattttatttgtaccattttaaatatgagaactaaagggttatttatgcataaattacctttatttgcctggtcagttttaataacagcagttctattactattatcactaccggtattggcaggagcaattactatgttaattactgacagaaattttaatacaaccttttttgatccagcaggaggaggagatcctgtattatatcaacatttattttgattttttggacatccagaagt

SEQ ID NO: 27 - DNA, 698 bp, Thalassiosira allenii
tcaacaaatcataaagatattggtacgctatacatgtggtttagttttttcatgttcctatttgcaggtgcgatggcactgggcatacgtgcagagctttttcagcctggactacagtttttagaaccagagcagtttaatagattgacgacactgcatggtctaatcatgatttttggtgccattatgccggcgtttgttggttttgctaactggatggtgccactaatgattggtgcgccagatatggccttccctaggctcaataatatgagtttctggttaatggtaccagctgcgattttactcattgcttcgatatttgtgccaggtggtgccatttcaggtggctggacgatgtatccaccactatctgtgcagaacacatctatgtcagtagacatgtctatcttggcattacatattttaggtgtgtcttctattgttggttcaattaatattattacaaccatcttgaacttacgcgcaccaggtatgacactaatgaaaatgccactgttcgtgtggacatggttgattacagcattcttgctgattgcagcaatgccagtgttggccggcgcgttaaccatgttgattactgatcgtcactttggtacagcgttctttaatgctgctggtggtggtgatccagtgttgttccaacatgtgttttggttttttggacatccagaagt

SEQ ID NO: 28 - DNA, 710 bp, Thalassiosira baltica
tcaacaaatcataaagatattggaactttgtacttaatatttggtgcaatttcaggtgttgcaggaacagcgttgtcgttatatattcgaattactttagcacaacctaatagtaattttttagagtataaccaccatttatataatgttattgtcacaggtcatgctatattaatgattttttttatggtaatgccaaccttaattggaggttttggcaactggtttgtgccattaatgattggggctccagatatggcattcccgcgaatgaacaatattagtttttggttactgccaccatcgttactattattgtttgcatcaatgttaacagaagctggtgtaggtactgggtgaactgtgtatccaccattatcgagtgcaactgctcattcaggaggttctgtagacctagcaatatttagtttacatctgtcaggtgcatcttctattctaggtgctattaattttatttgcacaatttttaatatgcgggtaaaaagtttatcattccataaccttcctttgtttgtttggtctgttttgattacagcatttttgttgttgttatctttaccagtattagcaggcgcaataacaatgttattaacagatcgaaattttaatactacttttttcgatcctgctggaggcggagatcctgtattatttcaacatcttttttgattttttggacatccagaagt

SEQ ID NO: 29 - DNA, 710 bp, Thalassiosira conferta
tcaacaaatcataaagatattggaactttatatttaatatttggagcaatatcaggtgttgcaggtactgcattatctttatatattcgaataactttagctcaaccaaatggtagttttttagaatataatcatcatttatataatgttattgtaactggacacgcaattttaatgattttttttatggtaatgcctaccttaattggaggatttggtaactggtttgtacctttaatgattggtgcacctgacatggcttttccaagaatgaataacattagcttttggctattaccaccttcgttattattattatttgcatcaatgttaactgaagcaggtgttggaaccggttggacagtatacccacctttatcaagtgcaacagctcattctggtggatctgtagatttagctatttttagtttacacgtgtctggaacttcgtctattctaggagcaatcaattttatttgtactatttttaatatgcgtgtaaaaagtttatctttccataatcttcctctatttgtatggtctgtactaattacagcgtttttattattattatcgttacccgtattagctggagcgattacaatgctattaactgatagaaattttaatactacgttttttgatcctgctggaggaggtgatcctgtactatttcaacatcttttctgattttttggacatccagaagt

SEQ ID NO: 30 - DNA, 710 bp, Thalassiosira decipiens
tcaacaaatcataaagatattggaactttgtacttaatatttggtgcaatttcaggtgttgcaggaacagcgttgtcgttatatattcgaattactttagcacaacctaatagtaattttttagagtataaccaccatttatataatgttattgtcacaggtcatgctatattaatgattttttttatggtaatgccaaccttaattggaggttttggcaactggtttgtgccattaatgattggggctccagatatggcattcccgcgaatgaacaatattagtttttggttactgccaccatcgttactattattgcttgcatcaatgttaacagaagctggtgtaggtactgggtgaactgtgtatccaccattatcgagtgcaactgctcattcaggaggttctgtagacctagcaatatttagtttacatctgtcaggtgcatcttctattctaggtgctattaattttatttgcacaatttttaatatgcgggtaaaaagtttatcattccataaccttcctttgtttgtttggtctgttttgattacagcatttttgttgttgttatctttaccagtattagcaggcgcaataacaatgttattaacagatcgaaattttaatactacttttttcgatcctgctggaggcggagatcctgtattatttcaacatcttttttgattttttggacatccagaagt

SEQ ID NO: 31 - DNA, 710 bp, Thalassiosira nordenskioldi
tcaacaaatcataaagatattggaactttatatttaatatttggagcaatatcaggggttgcaggtactgcattgtctttatatattcgaataactttagctcaaccaaatggtagttttttagaatataatcatcatttatacaatgttattgtaactggacacgcaattttaatgatttttttcatggtaatgcctaccttaattggaggatttggtaattggtttgtacctttaatgattggtgcacctgacatggcttttccaagaatgaataatattagtttttggttattaccaccttcgttactattattatttgcatcaatgttaactgaagcaggtgttggaaccggttggacagtatacccacccttatcaagtgcaacagctcattctggtggatctgtagatttagctatctttagtttgcacgtgtctggaacttcttctattctaggggcaatcaactttatctgtactatttttaatatgcgtgtaaaaagtctatctttccataatcttcctttatttgtatggtctgtactaattacagcgtttttattattattatcgttacctgtactagctggagcgatcacaatgttgttaactgatagaaattttaatactacgttttttgatcctgctggaggaggtgatcctgtactatttcaacatcttttctggttttttggacatccagaagt

SEQ ID NO: 32 - DNA, 710 bp, Thalassiosira ostupii
tcaacaaatcataaagatattggtacattatatttaatattcggtgcattcgcaggtgtaatgggtactactttctcagtacttattagaatggaattagctcaaccaggaagtcaaatccttcttggaaatggacaagtttataacgtaattattacagcacatgcttttttaatgattttctttatggttatgccaattttaattggtggatttggaaactggtttgtaccaattatgattggtgctccagatatggcctttcctagattaaataatattagtttttggttactaccaccttctttagttcttttattaggatcaggattagtagaagtaggagtaggtactggttggacagtgtatccttctttagcaagtattcaaagccactcgggtggtgcagtagacttagctatctttagcttacacttagctggtgtatcatcaatgttaggtgcaatgaacttcattacaactatctttaatatgagagctcctggtatgactctatataaaatgccattatttgtttgggcaacattaattacagcattcttacttttattatcattacctgttttagcaggaggtattacaatgttactaactgatagaaactttaatacttctttctttgacccagctggtggaggagatccaatattatatcaacatttattctggttttttggacatccagaaga

SEQ ID NO: 33 - DNA, 709 bp, Thalassiosira puntigera
tcaacaaatcataaagatattggaactttgtacttaatatttggtgcaatttcaggtgttgcaggaacagcgttgtcgttatatattcgaattactttagcacaacctaatagtaattttttagagtataaccaccatttatataatgttattgtcacaggtcatgctatattaatgattttttttatggtaatgccaaccttaattggaggttttggcaactggtttgtgccattaatgattggggctccagatatggcattcccgcgaatgaacaatattagtttttggttactgccaccatcgttactattattgtttgcatcaatgttaacagaagctggtgtaggtactgggtgaactgtgtatccaccattatcgagtgcaactgctcattcaggaggttctgtagacctagcaatatttagtttacatctgtcaggtgcatcttctattctaggtgctattaattttatttgcacaatttttaatatgcgggtaaaaagtttatcattccataaccttcctttgtttgtttggtctgttttgattacagcatttttgttgttgttatctttaccagtattagcaggcgcaataacaatgttattaacagatcgaaattttaatactacttttttcgatcctgctggaggcggagatcctgtattatttcaacatcttttttggttttttggacatccagaat

SEQ ID NO: 34 - DNA, 710 bp, Thalassiosira rotula
tcaacaaatcataaagatattggaactttgtacttaatatttggtgcaatttcaggtgttgcaggaacagcgttatcgttatatattcgaattactttagcacaacctaatagtaattttttagagtacaatcatcatttatataatgttattgtcacaggtcatgctatattaatgatttttttcatggtaatgcctacattaattggaggttttggtaactgatttgtcccgttaatgattggggcaccagatatggcattcccacgaatgaacaatattagtttttggttactgccaccatcgttattgctattgtttgcatcaatgttaacagaagctggtgtaggtactgggtgaactgtgtatccaccattgtctagtgcaactgctcactcaggaggttctgtagacttagcgatatttagtttacatctgtcaggtgcatcttctattctgggtgctattaattttatttgcaccatttttaatatgcgggtaaaaagtttatcattccataaccttcctttgtttgtttgatctgttttgattacagcatttttgttgttgctatctttaccagtattagcaggcgcaataacaatgttgttaacagatcgaaattttaatactactttttttgatcctgctgggggcggagatcctgtattatttcaacatcttttttgattttttggacatccagaagt

SEQ ID NO: 35 - DNA, 710 bp, Thalassiosira tenera
tcaacaaatcataaagatattggtactttatatctaatttttgcagcatttgctggtatcataggtacttttttttctgttattataagaatggaattatctttaccaggagatcaaattttaggaaataattatcaattatataatgttattataacagctcatgcttttattatgattttttttatggtaatgcctgcacttattggtggtttaggtaattggtttgtgcccttaatgataggtgctccagatatggcatttcctaggttaaataatataagtttttggttattacctccttctttttttttattattatcttcttctttagtagaagtaggggcaggtactggatggactgtttatccacctttagcaggtatacaaagtcattcaggaggttctgttgatttagctatttttagtttacatttagcaggagtatcttctcttttaggtgctattaattttattacaactgtaataaatatgcgaagtccagaaatggcttggcataaattatctttatttgtttggtctgttttcattacagcttttttattattattatctttacctgttttagcaggtgcaataactatgttgttaacagatagaaattttaatactactttttttgatccagcaggtggtggagatcctattttatatcaacatctattttgattttttggacatccagaagt

SEQ ID NO: 36 - DNA, 710 bp, Thalassiosira weissflogii
tcaacaaatcataaagatattggaactttatatttaatatttggagcaatatcaggggttgcaggtactgcattgtctttatatattcgaataactttagctcaaccaaatggtagttttttagaatataatcatcatttatacaatgttattgtaactggacacgcaattttaatgatttttttcatggtaatgcctaccttaattggaggatttggtaattggtttgtacctttaatgattggtgcacctgacatggcttttccaagaatgaataatattagtttttggttattaccaccttcgttactattattatttgcatcaatgttaactgaagcaggtgttggaaccggttggacagtatacccacctttatcaagtgcaacagctcattctggtggatctgtagatttagctatctttagtttgcacgtgtctggaacttcttctattctaggggcaatcaactttatctgtactatttttaatatgcgtgtaaaaagtctatctttccataatcttcctttatttgtatggtctgtactaattacagcgtttttattattattatcgttacctgtactagctggagcgatcacaatgttgttaactgatagaaattttaatactacgttttttgatcctgctggaggaggtgatcctgtactatttcaacatcttttctgattttttggacatccagaagt